SEQUENCE LISTING (46 sequences)

SEQ ID NO: 1 - protein, 207 aa, Homo sapiens
  SIGNAL 1..21: note = Signal sequence
MSPLLRRLLLAALLQLAPAQAPVSQPDAPGHQRKVVSWIDVYTRATCQPREVVVPLTVELMGTVAKQLVPSCVTVQRCGGCCPDDGLECVPTGQHQVRMQILMIRYPSSQLGEMSLEEHSQCECRPKKKDSAVKPDRAATPHHRPQPRSVPGWDSAPGAPSPADITHPTPAPGPSAHAAPSTTSALTPGPAAAAADAAASSVAKGGA

SEQ ID NO: 2 - protein, 188 aa, Homo sapiens [sequence omitted]

SEQ ID NO: 3 - protein, 119 aa, synthetic construct
  REGION 1..119: note = amino acid sequence from a VH of antibody 2H10
VQLQQPGTELVKPGASVKLSCKASGYTFTGFWIHWVKQRPGQGLEWIGHINPGNGGTNYNEKFKRMATLTVDKSSSTAYMQLSSLTSEDSAVYYCARSYSNYVRAMDYWGQGTSVTVSS

SEQ ID NO: 4 - protein, 106 aa, synthetic construct
  REGION 1..106: note = amino acid sequence from a VL of antibody 2H10
IQMTQTTSSLSASLGDRVTISCRASQDISNFLNWYQQKPDGTVKLLIYYTSTLHSGVPSRFSGSGSGTDYSLTISNLEQEDIATYFCQQGKTLPPTFGGGTKLEIK

SEQ ID NO: 5 - protein, 119 aa, synthetic construct
  REGION 1..119: note = amino acid sequence from a VH of a humanized form of antibody2H10
VQLVQSGAEVKKPGASVKVSCKASGYTFTGFWIHWVRQAPGQGLEWMGHINPGNGGTNYNEKFKRRVTMTRDKSISTAYMELSRLRSDDTAVYYCARSYSNYVRAMDYWGQGTLVTVSS

SEQ ID NO: 6 - protein, 107 aa, synthetic construct
  REGION 1..107: note = amino acid sequence of a VL of a humanized form of antibody 2H10
DIQMTQSPSSLSASVGDRVTITCRASQDISNFLNWYQQKPGKAPKLLIYYTSTLHSGVPSRFSGSGSGTDFTLTISSLQPEDFATYYCQQGKTLPPTFGGGTKVEIK

SEQ ID NO: 7 - protein, 116 aa, synthetic construct
  REGION 1..116: note = amino acid sequence from a VH of antibody 4E12
VQPQQPGAELVKPGASVKMSCKASGDTFTNSWIGWVTQRPGQGLEWIGDIFPGSGHTNYNEKFKNRATLTVDTSSSTAYMLLSSLTSDDSAVYYCVIENYAWFAYWGQGTLVTVSA

SEQ ID NO: 8 - protein, 107 aa, synthetic construct
  REGION 1..107: note = amino acid sequence of a VL of antibody 4E12
DIVMTQSQKFMSSSVGDRVSVTCKASQNVNTNVAWYQQKQGQSPRPLIYSASSRCSGVPDRFTGSGFGTDFTLTISNVQSEDLAEYFCQQYHSFPLTFGAGAKLDLK

SEQ ID NO: 9 - protein, 118 aa, synthetic construct
  REGION 1..118: note = amino acid sequence from a VH of antibody 2F5
VQLQQSGPELVKPGTSVRISCKASGYTFTTFYIHWVKQRPGQGLEWIGWFYPGNVNTNYNEKLKGKATLTADKSSSAAYLQLNSLTSEDSAVYFCTRSPYYGYVFDYWGQGTTLTVSS

SEQ ID NO: 10 - protein, 107 aa, synthetic construct
  REGION 1..107: note = amino acid sequence of a VL of antibody 2F5
DIVMTQSHKFMSTSVGDRVSITCKASQDVGTAVAWYQQKPGQSPKLLIYWASTRHTGVPDRFTGSGSGTDFTLTISNVQSEDLADYFCQQYSSSLTFGAGATKLELK

SEQ ID NO: 11 - DNA, 33 bp, synthetic construct
  misc_feature 1..33: note = Nucleotide sequence of 2H10 VL CDR1
  source 1..33: mol_type = other DNA
agggcaagtcaggacattagcaattttttaaac

SEQ ID NO: 12 - DNA, 21 bp, synthetic construct
  misc_feature 1..21: note = Nucleotide sequence of 2H10 VL CDR2
  source 1..21: mol_type = other DNA
tacacatcaacattacactca

SEQ ID NO: 13 - DNA, 27 bp, synthetic construct
  misc_feature 1..27: note = Nucleotide sequence of 2H10 VL CDR3
  source 1..27: mol_type = other DNA
caacagggtaaaacgcttcctcccacg

SEQ ID NO: 14 - DNA, 30 bp, synthetic construct
  misc_feature 1..30: note = Nucleotide sequence of 2H10 VH CDR1
  source 1..30: mol_type = other DNA
ggctacactttcactggcttctggatacac

SEQ ID NO: 15 - DNA, 51 bp, synthetic construct
  misc_feature 1..51: note = Nucleotide sequence of 2H10 VH CDR2
  source 1..51: mol_type = other DNA
catattaatcctggcaatggtggcactaactacaatgagaagttcaagaga

SEQ ID NO: 16 - DNA, 33 bp, synthetic construct
  misc_feature 1..33: note = Nucleotide sequence of 2H10 VH CDR3
  source 1..33: mol_type = other DNA
tcctatagtaactacgtgcgggctatggactac

SEQ ID NO: 17 - protein, 11 aa, synthetic construct
  REGION 1..11: note = Amino acid sequence of 2H10 VL CDR1
RASQDISNFLN

SEQ ID NO: 18 - protein, 7 aa, synthetic construct
  REGION 1..7: note = amino acid sequence of 2H10 VL CDR2
YTSTLHS

SEQ ID NO: 19 - protein, 9 aa, synthetic construct
  REGION 1..9: note = amino acid sequence of 2H10 VL CDR3
QQGKTLPPT

SEQ ID NO: 20 - protein, 10 aa, synthetic construct
  REGION 1..10: note = amino acid sequence of 2H10 VH CDR1
GYTFTGFWIH

SEQ ID NO: 21 - protein, 17 aa, synthetic construct
  REGION 1..17: note = amino acid sequence of 2H10 VH CDR2
HINPGNGGTNYNEKFKR

SEQ ID NO: 22 - protein, 11 aa, synthetic construct
  REGION 1..11: note = amino acid sequence of 2H10 VH CDR3
SYSNYVRAMDY

SEQ ID NO: 23 - DNA, 33 bp, synthetic construct
  misc_feature 1..33: note = nucleotide sequence of 2F5 VL CDR1
  source 1..33: mol_type = other DNA
aaggccagtcaggatgtgggtactgctgtagcc

SEQ ID NO: 24 - DNA, 21 bp, synthetic construct
  misc_feature 1..21: note = nucleotide sequence of 2F5 VL CDR2
  source 1..21: mol_type = other DNA
tgggcatccacccggcacact

SEQ ID NO: 25 - DNA, 24 bp, synthetic construct
  misc_feature 1..24: note = nucleotide sequence of 2F5 VL CDR3
  source 1..24: mol_type = other DNA
caacaatatagcagctctctcacg

SEQ ID NO: 26 - DNA, 30 bp, synthetic construct
  misc_feature 1..30: note = nucleotide sequence of 2F5 VH CDR1
  source 1..30: mol_type = other DNA
ggctacaccttcacaaccttctatatacac

SEQ ID NO: 27 - DNA, 51 bp, synthetic construct
  misc_feature 1..51: note = nucleotide sequence of 2F5 VH CDR2
  source 1..51: mol_type = other DNA
tggttttatcctggaaatgttaataccaactacaatgagaagctcaagggc

SEQ ID NO: 28 - DNA, 30 bp, synthetic construct
  misc_feature 1..30: note = nucleotide sequence of 2F5 VH CDR3
  source 1..30: mol_type = other DNA
tccccttactacggctacgtttttgactac

SEQ ID NO: 29 - protein, 11 aa, synthetic construct
  REGION 1..11: note = amino acid sequence of 2F5 VL CDR1
KASQDVGTAVA

SEQ ID NO: 30 - protein, 7 aa, synthetic construct
  REGION 1..7: note = amino acid sequence of 2F5 VL CDR2
WASTRHT

SEQ ID NO: 31 - protein, 8 aa, synthetic construct
  REGION 1..8: note = amino acid sequence of 2F5 VL CDR3
QQYSSSLT

SEQ ID NO: 32 - protein, 10 aa, synthetic construct
  REGION 1..10: note = amino acid sequence of 2F5 VH CDR1
GYTFTTFYIH

SEQ ID NO: 33 - protein, 17 aa, synthetic construct
  REGION 1..17: note = amino acid sequence of 2F5 VH CDR2
WFYPGNVNTNYNEKLKG

SEQ ID NO: 34 - protein, 10 aa, synthetic construct
  REGION 1..10: note = amino acid sequence of 2F5 VH CDR3
SPYYGYVFDY

SEQ ID NO: 35 - DNA, 33 bp, synthetic construct
  misc_feature 1..33: note = nucleotide sequence of 4E12 VL CDR1
  source 1..33: mol_type = other DNA
aaggccagtcagaatgtgaacactaatgtagcc

SEQ ID NO: 36 - DNA, 21 bp, synthetic construct
  misc_feature 1..21: note = nucleotide sequence of 4E12 VL CDR2
  source 1..21: mol_type = other DNA
tcggcatcctcccggtgcagt

SEQ ID NO: 37 - DNA, 27 bp, synthetic construct
  misc_feature 1..27: note = nucleotide sequence of 4E12 VL CDR3
  source 1..27: mol_type = other DNA
cagcaatatcacagctttccgctcacg

SEQ ID NO: 38 - DNA, 30 bp, synthetic construct
  misc_feature 1..30: note = nucleotide sequence of 4E12 VH CDR1
  source 1..30: mol_type = other DNA
ggcgacaccttcaccaactcctggataggc

SEQ ID NO: 39 - DNA, 51 bp, synthetic construct
  misc_feature 1..51: note = nucleotide sequence of 4E12 VH CDR2
  source 1..51: mol_type = other DNA
gatatttttcctgggagtggtcatactaactacaatgagaagttcaagaac

SEQ ID NO: 40 - DNA, 24 bp, synthetic construct
  misc_feature 1..24: note = nucleotide sequence of 4E12 VH CDR3
  source 1..24: mol_type = other DNA
gagaattatgcctggtttgcttat

SEQ ID NO: 41 - protein, 11 aa, synthetic construct
  REGION 1..11: note = amino acid sequence of 4E12 VL CDR1
KASQNVNTNVA

SEQ ID NO: 42 - protein, 7 aa, synthetic construct
  REGION 1..7: note = amino acid sequence of 4E12 VL CDR2
SASSRCS

SEQ ID NO: 43 - protein, 9 aa, synthetic construct
  REGION 1..9: note = amino acid sequence of 4E12 VL CDR3
QQYHSFPLT

SEQ ID NO: 44 - protein, 10 aa, synthetic construct
  REGION 1..10: note = amino acid sequence of 4E12 VH CDR1
GDTFTNSWIG

SEQ ID NO: 45 - protein, 17 aa, synthetic construct
  REGION 1..17: note = amino acid sequence of 4E12 VH CDR2
DIFPGSGHTNYNEKFKN

SEQ ID NO: 46 - protein, 8 aa, synthetic construct
  REGION 1..8: note = amino acid sequence of 4E12 VH CDR3
ENYAWFAY

(19) United States
(12) Patent Application Publication
(10) Pub. No.: US 2024/0002488 A1
(43) Pub. Date: Jan. 4, 2024

(54) METHOD OF TREATING OR PREVENTING LIVER CONDITIONS

(71) Applicants: CSL Limited, Parkville (AU); B-Creative Sweden AB, Balsta (SE)

(72) Inventors: Annika MEHLEM, Stockholm (SE); Ulf ERIKSSON, Stockholm (SE); Annelie FALKEVALL, Stockholm (SE); Isolde PALOMBO, Stockholm (SE)

(21) Appl. No.: 18/053,857

(22) Filed: Nov. 9, 2022

Related U.S. Application Data
(63) Continuation of application No. 16/095,021, filed on Oct. 19, 2018, now abandoned.

(30) Foreign Application Priority Data
Apr. 21, 2016 (AU) 2016901483
Apr. 21, 2016 (AU) 2016901494

(51) Int. Cl. C07K 16/22; A61K 31/7088; A61P 1/16; C12N 15/113

(52) U.S. Cl. CPC C07K 16/22; A61K 31/7088; A61K 2039/505; C12N 15/1136; A61P 1/16

(57) ABSTRACT

The present disclosure provides a method of treating a nonalcoholic fatty liver disease in a subject, the method comprising administering to the subject a compound that inhibits VEGF-B signaling.

### RELATED APPLICATION DATA

The present application is a continuation application of U.S. patent application Ser. No. 16/095,021, filed on Oct. 19, 2018, which claims priority from Australian Patent Application No. 2016901494 entitled “Method of Treating or Preventing Liver Conditions” filed 21 Apr. 2016 and from Australian Patent Application No. 2016901483 entitled “Method of Treating or Preventing Liver Conditions” filed 21 Apr. 2016. The entire contents of which are hereby incorporated by reference.

### INCORPORATION BY REFERENCE OF SEQUENCE LISTING

The Sequence Listing in the XML, named as 36719Z_SequenceListing.xml of 60 KB, created on Nov. 9, 2022, and submitted to the United States Patent and Trademark Office via Patent Center, is incorporated herein by reference.

### FIELD

The present application relates to a method for treating or preventing liver conditions.

### INTRODUCTION

Diseases characterized by fat accumulation in the liver are becoming increasingly prevalent in adults and children of industrialized countries due, in large part, to unhealthy eating habits and obesity. Alcoholic fatty liver disease (AFLD), which results from chronic excessive alcohol intake, essentially follows a pathological course involving steatosis, steatohepatitis (ASH), inflammation, cirrhosis, and, in some cases, hepatocellular carcinoma. Non-alcoholic fatty liver disease (NAFLD), considered the most common liver disease, is a term that encompasses a series of hepatic pathologies similar to those of AFLD that range in severity from hepatic steatosis (accumulation of fat in the liver), non-alcoholic steatohepatitis (NASH), cirrhosis, to hepatocellular carcinoma.

An estimated 30% of the U.S. population is affected with NAFLD. This prevalence increases to more than 60% in obese subjects and in subjects suffering from type 2 diabetes. NAFLD can progressively worsen from hepatic steatosis to NASH, which is characterized by the development of liver injury as evidenced by hepatocyte injury, infiltration of inflammatory cells and/or fibrosis. In turn, NASH can progress into liver cirrhosis, which is associated with the replacement of hepatocytes with scar tissue, and at more advanced stages, hepatocellular carcinoma. NAFLD is recognized as an important and common cause of cirrhosis and liver failure. NAFLD is also associated with an increased risk of cardiovascular disease. NAFLD is considered to be part of the metabolic syndrome. Similar to those with metabolic syndrome, >80% of individuals with NAFLD are overweight, with approximately 30% being obese. Furthermore, those with NAFLD also often have concurrent hyperlipidemia, type 2 diabetes mellitus (T2DM), and are hypertensive.

There are currently no effective treatments for NAFLD, and most treatments hinge on managing associated conditions such as obesity, diabetes mellitus, and hyperlipidemia. Other therapies for NAFLD mainly target lifestyle habits, such as exercise and diet. Pharmacological interventions aimed at targeting weight loss and insulin resistance have limited efficacy. For example, studies of metformin and statins have shown a lack of effect in improving the liver histology in patients with NAFLD or NASH.

### SUMMARY

In producing the present invention, the inventors studied the effects of inhibiting VEGF-B in an accepted model of NAFLD and NASH. The inventors studied the effects of inhibiting expression of VEGF-B in a knockout mouse fed on a high fat diet or fed a choline-deficient high fat diet, thus demonstrating the prophylactic effects of inhibiting this protein. The inventors also administered an antagonistic antibody to mice fed on either a high fat diet or a choline-deficient high fat diet to study the therapeutic effects of inhibiting this protein. In all cases, VEGF-B inhibition reduces or protects against lipid accumulation in the liver, with all approaches reducing or maintaining lipids at a level similar to control animals. Thus, inhibition of VEGF-B reduces lipid accumulation to a normal or healthy level, rather than to potentially harmfully low levels. Inhibition of VEGF-B also reduced or prevented liver inflammation and several characteristics of NASH, including hepatocellular ballooning, one of the main pathologies of NASH, Mallory-Denk body (MDB) formation, inflammatory foci and satellitosis. Accordingly, the inventors have shown that they can prevent or treat NAFLD and prevent development of or treat NASH. Clearly, such methods have additional benefits in preventing cirrhosis, hepatic fibrosis and/or hepatocellular carcinoma.

The findings by the inventors provide the basis for methods for treating or preventing NAFLD or a complication thereof in a subject by inhibiting VEGF-B signaling. For example, the present disclosure provides a method for treating or preventing NAFLD or a complication thereof in a subject, the method comprising administering to the subject a compound that inhibits VEGF-B signaling.

In one example, the NAFLD is hepatic steatosis, for example, isolated hepatic steatosis.

In one example, the NAFLD is NASH.

In one example, the NAFLD is cirrhosis.

In one example, the NAFLD is NASH-derived cirrhosis.

In another example, the NAFLD is NASH-associated cirrhosis.

In another example, the NAFLD is NASH-associated hepatic fibrosis.

In one example, the present disclosure provides a method for preventing or delaying the onset of cirrhosis in a subject suffering from a NAFLD, the method comprising administering to the subject a compound that inhibits VEGF-B signaling.

For example, the subject suffers from NASH and the method prevents or delays the onset of cirrhosis.

In one example, the present disclosure provides a method for delaying the progression of cirrhosis in a subject suffering from a NAFLD, the method comprising administering to the subject a compound that inhibits VEGF-B signaling.

In one example, the present disclosure provides a method for delaying the progression of a NAFLD to cirrhosis in a subject suffering from a NAFLD, the method comprising administering to the subject a compound that inhibits VEGF-B signaling.

In one example, the complication of the NAFLD is hepatocellular carcinoma.

In one example, the hepatocellular carcinoma is NASH-derived hepatocellular carcinoma.

In another example, the hepatocellular carcinoma is NASH-associated hepatocellular carcinoma.

In one example, the present disclosure provides a method for preventing or delaying the onset of hepatocellular carcinoma in a subject suffering from a NAFLD, the method comprising administering to the subject a compound that inhibits VEGF-B signaling.

For example, the subject suffers from NASH and the method prevents or delays the onset of hepatocellular carcinoma.

In one example, the present disclosure provides a method for delaying the progression of hepatocellular carcinoma in a subject suffering from a NAFLD, the method comprising administering to the subject a compound that inhibits VEGF-B signaling.

In one example, the present disclosure provides a method for delaying the progression of a NAFLD to hepatocellular carcinoma in a subject suffering from a NAFLD, the method comprising administering to the subject a compound that inhibits VEGF-B signaling.

In one example, the subject suffers from a NAFLD and the method treats said disease. For example, the subject suffers from hepatic steatosis. For example, the subject suffers from NASH.

In one example, the subject suffering from the NAFLD is additionally overweight or obese and/or suffers from diabetes, e.g., type 2 diabetes and/or suffers from metabolic syndrome.

In one example, the disclosure provides a method for treating or preventing NAFLD or a complication thereof in an overweight or obese subject, the method comprising administering to the subject a compound that inhibits VEGF-B signaling.

In one example, administering the compound does not substantially or significantly reduce the weight of the subject compared to the subject's weight prior to the administration.

In one example, the subject suffers from a NAFLD (e.g., as described above) and the method prevents or slows progression of the disease. For example, the subject suffers from steatosis and the method slows or prevents progression to NASH. In another example, the subject suffers from NASH and the method slows or prevents progression to cirrhosis.

In one example, the subject suffers from a NAFLD (e.g., as described above) and the method prevents or reduces the risk of developing a complication of the NAFLD. For example, the subject suffers from a NAFLD and the method prevents development of or reduces the risk of hepatocellular carcinoma.

In one example, a subject suffering from a NAFLD is diagnosed based on one or more of elevated serum levels of a liver enzyme, e.g., alanine aminotransferase (ALAT) or aspartate aminotransferase (ASAT), a liver ultrasound or a liver biopsy.

In one example, the subject is at risk of developing a NAFLD, e.g., suffers from a comorbidity of a NAFLD. For example, the subject is obese and/or suffers from diabetes, e.g., type 2 diabetes and/or suffers from metabolic syndrome.

In one example, the compound is administered in an amount effective to have one or more of the following effects:
Reduce or prevent lipids, e.g., neutral lipids accumulating in the liver of a subject, e.g., as assessed in a liver biopsy;
Reduce or prevent inflammation in the liver of the subject, e.g., by reducing the number of immune cells in the liver of the subject;
Reduce or prevent development of pathologic changes of NAFLD, such as, Mallory-Denk bodies or hepatocyte ballooning or inflammatory foci or satellitosis in the liver of a subject;
Reduce or prevent development of hepatic fibrosis and/or cirrhosis;
Reduce or prevent development of hepatocellular carcinoma.

In one example, the present disclosure provides a method for reducing the level of lipids, e.g., neutral lipids, in the liver of a subject suffering from NAFLD (e.g., suffering from NASH), the method comprising administering to the subject a compound that inhibits VEGF-B signaling. In one example, the level of lipids is reduced compared to the level in the subject prior to administration of the compound or compared to the level observed in a population of subjects suffering from the NAFLD. In another example, the level of lipids is reduced to a level similar to (e.g., with 10% of) or the same as a subject not suffering from NAFLD or a population of subjects not suffering from NAFLD.

The present disclosure also provides a method for reducing inflammation in the level of a subject suffering from NAFLD the method comprising administering to the subject a compound that inhibits VEGF-B signaling.

In one example, the compound that inhibits VEGF-B signaling specifically inhibits VEGF-B signaling. This does not mean that a method of the present disclosure does not encompass inhibiting signaling of multiple VEGF proteins, only that the compound (or part thereof) that inhibits VEGF-B signaling is specific to VEGF-B, e.g., is not a general inhibitor of VEGF proteins. This term also does not exclude, e.g., a bispecific antibody or protein comprising binding domains thereof, which can specifically inhibit VEGF-B signaling with one (or more) binding domains and can specifically inhibit signaling of another protein with another binding domain.

In one example, a compound that inhibits VEGF-B signaling binds to VEGF-B.

For example, the compound is a protein comprising an antibody variable region that binds to or specifically binds to VEGF-B and neutralizes VEGF-B signaling.

In one example, the compound is an antibody mimetic. For example, the compound is a protein comprising an antigen binding domain of an immunoglobulin, e.g., an IgNAR, a camelid antibody or a T cell receptor.

In one example, a compound is a domain antibody (e.g., comprising only a heavy chain variable region or only a light chain variable region that binds to VEGF-B) or a heavy chain only antibody (e.g., a camelid antibody or an IgNAR) or variable region thereof.

In one example, a compound is a protein comprising a Fv. For example, the protein is selected from the group consisting of:
(i) a single chain Fv fragment (scFv);
(ii) a dimeric scFv (di-scFv); or
(iv) a diabody;
(v) a triabody;
(vi) a tetrabody;
(vii) a Fab;
(viii) a F(ab′)₂;
(ix) a Fv; or
(x) one of (i) to (ix) linked to a constant region of an antibody, Fc or a heavy chain constant domain (C_{H}) 2 and/or C_{H}3.

In another example, a compound is an antibody. Exemplary antibodies are full-length and/or naked antibodies.

In one example, the compound is a protein that is recombinant, chimeric, CDR grafted, humanized, synhumanized, primatized, deimmunized or human.

In one example, the compound is a protein comprising an antibody variable region that competitively inhibits the binding of antibody 2H10 to VEGF-B. In one example, the protein comprises a heavy chain variable region (V_{H}) comprising a sequence set forth in SEQ ID NO: 3 and a light chain variable region (V_{L}) comprising a sequence set forth in SEQ ID NO: 4.

In one example, the compound is a protein comprising a humanized variable region of antibody 2H10. For example, the protein comprises a variable region comprising the complementarity determining regions (CDRs) of the V_{H} and/or the V_{L} of antibody 2H10. For example, the protein comprises:
(i) a V_{H} comprising:
   (a) a CDR1 comprising a sequence set forth in amino acids 25-34 of SEQ ID NO: 3;
   (b) a CDR2 comprising a sequence set forth in amino acids 49-65 of SEQ ID NO: 3; and
   (c) a CDR3 comprising a sequence set forth in amino acids 98-108 of SEQ ID NO: 3; and/or
(ii) a V_{L} comprising:
   (a) a CDR1 comprising a sequence set forth in amino acids 23-33 of SEQ ID NO: 4;
   (b) a CDR2 comprising a sequence set forth in amino acids 49-55 of SEQ ID NO: 4; and
   (c) a CDR3 comprising a sequence set forth in amino acids 88-96 of SEQ ID NO: 4.

In one example, the compound is a protein comprising a V_{H} and a V_{L}, the V_{H} and V_{L} being humanized variable regions of antibody 2H10. For example, the protein comprises:
(i) a V_{H} comprising:
   (a) a CDR1 comprising a sequence set forth in amino acids 25-34 of SEQ ID NO: 3;
   (b) a CDR2 comprising a sequence set forth in amino acids 49-65 of SEQ ID NO: 3; and
   (c) a CDR3 comprising a sequence set forth in amino acids 98-108 of SEQ ID NO: 3; and
(ii) a V_{L} comprising:
   (a) a CDR1 comprising a sequence set forth in amino acids 23-33 of SEQ ID NO: 4;
   (b) a CDR2 comprising a sequence set forth in amino acids 49-55 of SEQ ID NO: 4; and
   (c) a CDR3 comprising a sequence set forth in amino acids 88-96 of SEQ ID NO: 4.

In one example, the variable region or V_{H} in any of the foregoing paragraphs comprises a sequence set forth in SEQ ID NO: 5.

In one example, the variable region or V_{L} in any of the foregoing paragraphs comprises a sequence set forth in SEQ ID NO: 6.

In one example, the compound is an antibody.

In one example, the compound is an antibody comprising a V_{H} comprising a sequence set forth in SEQ ID NO: 5 and a V_{L} comprising a sequence set forth in SEQ ID NO: 6.

In one example, the protein or antibody is any form of the protein or antibody encoded by a nucleic acid encoding any of the foregoing proteins or antibodies.

In one example, the protein or antibody comprises:
(i) a V_{H} comprising:
   (a) a CDR1 comprising a sequence encoded by a nucleic acid comprising SEQ ID NO: 11 or comprising an amino acid sequence of SEQ ID NO: 17;
   (b) a CDR2 comprising a sequence encoded by a nucleic acid comprising SEQ ID NO: 12 or comprising an amino acid sequence of SEQ ID NO: 18; and
   (c) a CDR3 comprising a sequence encoded by a nucleic acid comprising SEQ ID NO: 13 or comprising an amino acid sequence of SEQ ID NO: 19; and/or
(ii) a V_{L} comprising:
   (a) a CDR1 comprising a sequence encoded by a nucleic acid comprising SEQ ID NO: 14 or comprising an amino acid sequence of SEQ ID NO: 20;
   (b) a CDR2 comprising a sequence encoded by a nucleic acid comprising SEQ ID NO: 15 or comprising an amino acid sequence of SEQ ID NO: 21; and
   (c) a CDR3 comprising a sequence encoded by a nucleic acid comprising SEQ ID NO: 16 or comprising an amino acid sequence of SEQ ID NO: 22.

In one example, the protein or antibody comprises:
(i) a V_{H} comprising:
   (a) a CDR1 comprising a sequence encoded by a nucleic acid comprising SEQ ID NO: 23 or comprising an amino acid sequence of SEQ ID NO: 29;
   (b) a CDR2 comprising a sequence encoded by a nucleic acid comprising SEQ ID NO: 24 or comprising an amino acid sequence of SEQ ID NO: 30; and
   (c) a CDR3 comprising a sequence encoded by a nucleic acid comprising SEQ ID NO: 25 or comprising an amino acid sequence of SEQ ID NO: 31; and/or
(ii) a V_{L} comprising:
   (a) a CDR1 comprising a sequence encoded by a nucleic acid comprising SEQ ID NO: 26 or comprising an amino acid sequence of SEQ ID NO: 32;
   (b) a CDR2 comprising a sequence encoded by a nucleic acid comprising SEQ ID NO: 27 or comprising an amino acid sequence of SEQ ID NO: 33; and
   (c) a CDR3 comprising a sequence encoded by a nucleic acid comprising SEQ ID NO: 28 or comprising an amino acid sequence of SEQ ID NO: 34.

In one example, the protein or antibody comprises:
(i) a V_{H} comprising:
   (a) a CDR1 comprising a sequence encoded by a nucleic acid comprising SEQ ID NO: 35 or comprising an amino acid sequence of SEQ ID NO: 41;
   (b) a CDR2 comprising a sequence encoded by a nucleic acid comprising SEQ ID NO: 36 or comprising an amino acid sequence of SEQ ID NO: 42; and
   (c) a CDR3 comprising a sequence encoded by a nucleic acid comprising SEQ ID NO: 37 or comprising an amino acid sequence of SEQ ID NO: 43; and/or
(ii) a V_{L} comprising:
   (a) a CDR1 comprising a sequence encoded by a nucleic acid comprising SEQ ID NO: 38 or comprising an amino acid sequence of SEQ ID NO: 44;
   (b) a CDR2 comprising a sequence encoded by a nucleic acid comprising SEQ ID NO: 39 or comprising an amino acid sequence of SEQ ID NO: 45; and
   (c) a CDR3 comprising a sequence encoded by a nucleic acid comprising SEQ ID NO: 40 or comprising an amino acid sequence of SEQ ID NO: 46.

In one example, the compound is within a composition. For example, the composition comprises a protein comprising an antibody variable region or a V_{H} or a V_{L} or an antibody as described herein. In one example, the composition additionally comprises one or more variants of the protein or antibody. For example, that comprises a variant missing an encoded C-terminal lysine residue, a deamidated variant and/or a glycosylated variant and/or a variant comprising a pyroglutamate, e.g., at the N-terminus of a protein and/or a variant lacking a N-terminal residue, e.g., a N-terminal glutamine in an antibody or V region and/or a variant comprising all or part of a secretion signal. Deamidated variants of encoded asparagine residues may result in isoaspartic, and aspartic acid isoforms being generated or even a succinamide involving an adjacent amino acid residue. Deamidated variants of encoded glutamine residues may result in glutamic acid. Compositions comprising a heterogeneous mixture of such sequences and variants are intended to be included when reference is made to a particular amino acid sequence.

In one example, the compound that inhibits VEGF-B signaling inhibits or prevents expression of VEGF-B. For example, the compound is selected from the group an antisense, a siRNA, a RNAi, a ribozyme and a DNAzyme.

In one example, the VEGF-B is mammalian VEGF-B, e.g., human VEGF-B.

In one example, the subject is a mammal, for example a primate, such as a human.

Methods of treatment described herein can additionally comprise administering a further compound to treat or prevent the NAFLD.

Methods of treatment of NAFLD described herein can additionally comprise administering a further compound to treat or prevent (or delay progression of) obesity. Exemplary compounds are described herein.

The present disclosure also provides a compound that inhibits VEGF-B signaling for use in the treatment or prevention of a NAFLD or a complication thereof.

The present disclosure also provides for use of a compound that inhibits VEGF-B signaling in the manufacture of a medicament for treating or preventing a NAFLD or a complication thereof.

The present disclosure also provides a kit comprising a compound that inhibits VEGF-B signaling packaged with instructions for use in the treatment or prevention of a NAFLD or a complication thereof.

Exemplary NAFLDs and complications thereof and compounds are described herein and are to be taken to apply mutatis mutandis to the examples of the disclosure set out in the previous three paragraphs.

### BRIEF DESCRIPTION OF THE DRAWINGS

The patent or application file contains at least one drawing executed in color. Copies of this paper or patent application publication with color drawing(s) will be provided by the Office upon request and payment of the necessary fee.

FIG. 1 is a graphical representation showing A. blood glucose levels, B. bodyweight and C. serum alanine aminotransferase levels in chow-fed WT, HFD-fed WT and HFD-fed Vegfb^{-/-} mice. Values are mean±s.e.m. ####P<0.0001 compared to chow-fed WT. *P<0.05, compared to HFD-fed WT mice. N=3-8/group (A), n=3-8/group (B), n=6-10/group (C).

FIG. 2 is a graphical representation showing quantification of A. Oil red O staining of livers sections and B. relative hepatic mRNA expression of fatty acid synthase (fasn) in chow-fed WT, HFD-fed WT and HFD-fed Vegfb^{-/-} mice. Values are mean±s.e.m. ###P<0.001 compared to chow-fed WT. *P<0.05, compared to HFD-fed WT mice. N=6-11/group (A), n=3-6/group (B).

FIG. 3 is a graphical representation showing quantification of A. adipophilin, B. mannose-6-phosphate receptor binding protein 1 (tip47) in liver sections and C. relative hepatic mRNA expression of adipophilin (plin2a) in chow-fed WT, HFD-fed WT and HFD-fed Vegfb^{-/-} mice. Values are mean±s.e.m. ####P<0.0001 compared to chow-fed WT. ****P<0.0001 compared to HFD-fed WT mice. N=4-11/group (A), n=5-8/group (B), n=3-6/group (C).

FIG. 4 is a graphical representation showing quantification of A. protein tyrosine phosphatase, receptor type, C (CD45), B. EGF-like module-containing mucin-like hormone receptor-like 1 (F4/80) in liver sections and C. relative hepatic mRNA expression of monocyte chemoattractant protein-1 (mcp1) in chow-fed WT, HFD-fed WT and HFD-fed Vegfb^{-/-} mice. Values are mean±s.e.m. ##P<0.01, ###P<0.001 compared to chow-fed WT. *P<0.05, **P<0.01, ****P<0.0001 compared to HFD-fed WT mice. N=4-6/group (A), n=4-8/group (B), n=4-5/group (C).

FIG. 5 is a graphical representation showing quantification of the total number of all identified ballooned hepatocytes, Mallory-Denk bodies, inflammatory foci and satellitosis in liver sections of chow-fed WT, HFD-fed WT and HFD-fed Vegfb^{-/-} mice. Values are mean±s.e.m. ####P<0.0001 compared to chow-fed WT and ****P<0.0001 compared to HFD-fed WT mice. N=5-9/group.

FIG. 6 is a graphical representation showing A. blood glucose levels, B. bodyweight, C. liver weight, D. ratio of liver weight and body weight and E. serum alanine aminotransferase levels in chow-fed WT and HFD-fed mice treated with anti-VEGF antibody (2H10) or control antibody. Values are mean±s.e.m. ####P<0.0001 compared to chow-fed WT. n=6-10/group.

FIG. 7 is a graphical representation showing quantification of A. Oil red O staining in liver sections and B. relative hepatic mRNA expression of fatty acid synthase (fasn) in chow-fed WT and HFD-fed mice treated with anti-VEGF antibody (2H10) or control antibody. Values are means±s.e.m. ####P<0.001 compared to chow-fed WT. **P<0.01, compared to control treated HFD-fed mice. N=6-11 (A), n=3-6 (B).

FIG. 8 is a graphical representation showing quantification of A. adipophilin, B. mannose-6-phosphate receptor binding protein 1 (tip47) in liver sections and C. relative hepatic mRNA expression of adipophilin (plin2a) in chow-fed WT and HFD-fed mice treated with anti-VEGF antibody (2H10) or control antibody. Values are mean±s.e.m. ####P<0.0001 compared to chow-fed WT. **P<0.01, ****P<0.0001 compared to control treated HFD-fed mice. N=5-11/group (A), n=6-8/group (B), n=3-5/group (C).

FIG. 9 is a graphical representation showing quantification of A. protein tyrosine phosphatase, receptor type, C (CD45), B. EGF-like module-containing mucin-like hormone receptor-like 1 (F4/80) in liver sections and C. relative hepatic mRNA expression of monocyte chemoattractant protein-1 (mcp1) in chow-fed WT and HFD-fed mice treated with anti-VEGF antibody (2H10) or control antibody. Values are mean±s.e.m. ##P<0.01, ###P<0.001 compared to chow-fed WT. **P<0.01, ****P<0.0001 compared to control treated HFD-fed mice. N=4-7/group (A), n=4-8/group (B), n=4-7/group (C).

FIG. 10 is a graphical representation showing quantification of the total number of all identified ballooned hepatocytes, Mallory-Denk bodies, inflammatory foci and satellitosis in liver sections of chow-fed WT and HFD-fed mice treated with anti-VEGF antibody (2H10) or control antibody. Values are mean±s.e.m. ####P<0.0001 compared to chow-fed WT and ****P <0.0001 compared to control treated HFD-fed mice. N=5-9/group.

FIG. 11 is a graphical representation showing A. bodyweight and blood glucose levels, B intraperitoneal glucose and C. intraperitoneal insulin tolerance tests (IPGTTs and IPITTs) with quantifications shown as AUC analysis in WT, Vegfb^{+/-} and Vegfb^{-/-} mice on short-term CD diet (CD diet for 5 months; CD5). Values are means±s.e.m. *P<0.05, compared to WT on CD diet. N=4-13/group.

FIG. 12 is a graphical representation showing A. liver weight and ratio of liver weight to body weight, and B. quantification of serum alanine aminotransferase (ALAT) levels in WT, Vegfb^{+/-} and Vegfb^{-/-} mice on CD5 diet. Values are means±s.e.m. *P<0.05, ***P<0.001 compared to WT mice on CD diet. N=4-13/group.

FIG. 13 is a graphical representation showing A. quantification of Oil red O (ORO) staining of liver sections and B. quantification of plasma levels of triglycerides (TGs), ketones (KBs) and non-esterified fatty acids (NEFAs) in WT, Vegfb^{+/-} and Vegfb^{-/-} mice on CD5 diet. Values are means±s.e.m. *P<0.05, **P<0.01, ***P<0.001 compared to WT mice on CD diet. N=4-13/group.

FIG. 14 is a graphical representation showing quantification of immunolabelling of A. protein tyrosine phosphatase, receptor type, C (CD45) and B. EGF-like module-containing mucin-like hormone receptor-like 1 (F4/80) in liver sections from WT, Vegfb^{+/-} and Vegfb^{-/-} mice on CD5 diet. Values are means±s.e.m. *P<0.05, **P<0.01, ***P<0.001 compared to WT on CD diet. N=4-13/group.

FIG. 15 is a graphical representation showing A. bodyweight and blood glucose levels, B. intraperitoneal glucose (IPGTT) and C. intraperitoneal insulin tolerance tests (IPITT) with quantifications shown as area-under-curve analysis in chow-fed WT mice and WT mice on CD5 diet treated with anti-VEGF antibody (2H10) or control antibody. Values are means±s.e.m. #P<0.05, compared to chow-fed WT. n=8-10/group.

FIG. 16 is a graphical representation showing A. liver weight and the ratio of liver weight to body weight and B. quantification of serum alanine aminotransferase (ALAT) levels in chow-fed WT mice and WT mice on CD5 diet treated with anti-VEGF antibody (2H10) or control antibody. Values are means±s.e.m. ##P<0.01, ###P<0.001 compared to chow-fed WT mice. **P<0.01, compared to control treated C57BL/6 mice on CD diet. N=8-10/group.

FIG. 17 is a graphical representation showing A. quantification of ORO staining and B. quantification of plasma levels of triglycerides (TGs), ketones (KBs) and non-esterified fatty acids (NEFAs) in chow-fed WT mice and WT mice on CD5 diet treated with anti-VEGF antibody (2H10) or control antibody. Values are means±s.e.m. #P<0.05, ##P<0.01, ###P<0.001 compared to chow-fed WT mice. *P<0.05, ****P<0.0001, compared control treated mice on CD diet. N=8-10/group.

FIG. 18 is a graphical representation showing A. quantification of adipophilin expression in liver sections of chow-fed WT mice and WT mice on CD5 diet treated with anti-VEGF antibody (2H10) or control antibody. Values are means±s.e.m. ####P<0.0001 compared to chow-fed WT animals. ****P<0.0001 compared to control treated mice on CD diet. N=8-10/group.

FIG. 19 is a graphical representation showing quantification of immunolabelling of A. protein tyrosine phosphatase, receptor type, C (CD45) and B. EGF-like module-containing mucin-like hormone receptor-like 1 (F4/80) in liver sections of chow-fed WT mice and WT mice on CD5 diet treated with anti-VEGF antibody (2H10) or control antibody. Values are means±s.e.m. ##P<0.01, ###P<0.001 compared to chow-fed WT mice. **P<0.01, ***P<0.001 compared to control treated mice on CD diet. N=8-10/group.

FIG. 20 is a graphical representation showing quantification of hepatic fibrosis using Masson trichrome staining of liver sections from chow-fed WT mice and WT mice on CD5 diet treated with anti-VEGF antibody (2H10) or control antibody. Values are means±s.e.m. ####P<0.001 compared to chow-fed WT mice. ***P<0.001 compared to control treated mice on CD diet. N=8-10/group.

FIG. 21 is a graphical representation showing total hepatic scoring of NASH in H&E stained liver sections of chow-fed WT mice and WT mice on CD5 diet treated with anti-VEGF antibody (2H10) or control antibody Values are means s.e.m. ####P<0.0001 compared to chow-fed animals and ***P<0.001 compared to C57BL/6 mice on CD diet control treated WT mice. N=5-10/group.

FIG. 22 is a graphical representation showing A. bodyweight and blood glucose levels, B. intraperitoneal glucose and C. intraperitoneal insulin tolerance tests (IPGTTs and IPITTs) with quantifications shown as AUC analysis in chow-fed WT mice and WT mice on long-term CD diet (CD diet for 12 months; CD12) treated with anti-VEGF antibody (2H10) or control antibody. Values are means±s.e.m. ##P<0.01, ###P<0.001 compared to chow-fed WT mice. N=8/group.

FIG. 23 is a series of graphical representations of WT chow fed mice and WT mice on CD12 diet treated with anti-VEGF antibody or control antibody showing A. liver weight and ratio of liver weight to body weight, B. quantification of serum alanine aminotransferase (ALAT) levels, C. a representative image of a gadolinium-enhanced MRI scan. The tumor burden is indicated by arrow. D. A graph summarizing mice treated with 2H10 or control antibody without tumor and with tumor on long-term CD diet. Symbols depict individual mice. E. Representative images of macroscopy of livers, F. liver tumor site in mice treated with control antibody on CD12 diet. Values are means±s.e.m. ##P<0.01, ###P<0.001 compared to chow-fed WT mice. *P<0.05, **P<0.01, compared to C57BL/6 mice on CD diet control treated mice. N=8/group (A-E); n=4 (F) FIG. 24 is a graphical representation showing quantification of ORO staining in livers of chow-fed WT and WT mice on CD12 diet treated with anti-VEGF antibody or control antibody with tumours or without tumours. Values are means±s.e.m. #P<0.01, ##P<0.01, ###P<0.001 compared to chow-fed WT mice. ****P<0.0001, compared to control treated mice on CD diet without tumors. N=4-8/group.

FIG. 25 is a graphical representation showing quantification of immunolabelling of A. protein tyrosine phosphatase, receptor type, C (CD45) and B. EGF-like module-containing mucin-like hormone receptor-like 1 (F4/80) in liver sections of chow-fed WT mice and WT mice on CD12 diet treated with anti-VEGF (2H10) antibody or control antibody treated mice with tumours or without tumours. Values are means±s.e.m. #P<0.01, ###P<0.001 compared to chow-fed WT mice. *P<0.01, compared to control treated mice on CD diet without tumors. \hat{ }\hat{ }\hat{ }P<0.001 compared control treated mice on CD diet with tumors. N=4-8/group.

### Key to Sequence Listing

SEQ ID NO: 1 is an amino acid sequence of a human VEGF-B₁₈₆ isoform containing a 21 amino acid N-terminal signal sequence

SEQ ID NO: 2 is an amino acid sequence of a human VEGF-B₁₆₇ isoform containing a 21 amino acid N-terminal signal sequence

SEQ ID NO: 3 is an amino acid sequence from a V_{H} of antibody 2H10.

SEQ ID NO: 4 is an amino acid sequence from a V_{L} of antibody 2H10.

SEQ ID NO: 5 is an amino acid sequence from a V_{H} of a humanized form of antibody 2H10.

SEQ ID NO: 6 is an amino acid sequence of a V_{L} of a humanized form of antibody 2H10.

SEQ ID NO: 7 is an amino acid sequence from a V_{H} of antibody 4E12.

SEQ ID NO: 8 is an amino acid sequence of a V_{L} of antibody 4E12.

SEQ ID NO: 9 is an amino acid sequence from a V_{H} of antibody 2F5.

SEQ ID NO: 10 is an amino acid sequence of a V_{L} of antibody 2F5.

SEQ ID NO: 11 is a nucleotide sequence from a V_{L} CDR1 of antibody 2H10

SEQ ID NO: 12 is a nucleotide sequence from a V_{L} CDR2 of antibody 2H10

SEQ ID NO: 13 is a nucleotide sequence from a V_{L} CDR3 of antibody 2H10

SEQ ID NO: 14 is a nucleotide sequence from a V_{H} CDR1 of antibody 2H10

SEQ ID NO: 15 is a nucleotide sequence from a V_{H} CDR2 of antibody 2H10

SEQ ID NO: 16 is a nucleotide sequence from a V_{H} CDR3 of antibody 2H10

SEQ ID NO: 17 is an amino acid sequence from a V_{L} CDR1 of antibody 2H10

SEQ ID NO: 18 is an amino acid sequence from a V_{L} CDR2 of antibody 2H10

SEQ ID NO: 19 is an amino acid sequence from a V_{L} CDR3 of antibody 2H10

SEQ ID NO: 20 is an amino acid sequence from a V_{H} CDR1 of antibody 2H10

SEQ ID NO: 21 is an amino acid sequence from a V_{H} CDR2 of antibody 2H10

SEQ ID NO: 22 is an amino acid sequence from a V_{H} CDR3 of antibody 2H10

SEQ ID NO: 23 is a nucleotide sequence from a V_{L} CDR1 of antibody 2F5

SEQ ID NO: 24 is a nucleotide sequence from a V_{L} CDR2 of antibody 2F5

SEQ ID NO: 25 is a nucleotide sequence from a V_{L} CDR3 of antibody 2F5

SEQ ID NO: 26 is a nucleotide sequence from a V_{H} CDR1 of antibody 2F5

SEQ ID NO: 27 is a nucleotide sequence from a V_{H} CDR2 of antibody 2F5

SEQ ID NO: 28 is a nucleotide sequence from a V_{H} CDR3 of antibody 2F5

SEQ ID NO: 29 is an amino acid sequence from a V_{L} CDR1 of antibody 2F5

SEQ ID NO: 30 is an amino acid sequence from a V_{L} CDR2 of antibody 2F5

SEQ ID NO: 31 is an amino acid sequence from a V_{L} CDR3 of antibody 2F5

SEQ ID NO: 32 is an amino acid sequence from a V_{H} CDR1 of antibody 2F5

SEQ ID NO: 33 is an amino acid sequence from a V_{H} CDR2 of antibody 2F5

SEQ ID NO: 34 is an amino acid sequence from a V_{H} CDR3 of antibody 2F5

SEQ ID NO: 35 is a nucleotide sequence from a V_{L} CDR1 of antibody 4E12

SEQ ID NO: 36 is a nucleotide sequence from a V_{L} CDR2 of antibody 4E12

SEQ ID NO: 37 is a nucleotide sequence from a V_{L} CDR3 of antibody 4E12

SEQ ID NO: 38 is a nucleotide sequence from a V_{H} CDR1 of antibody 4E12

SEQ ID NO: 39 is a nucleotide sequence from a V_{H} CDR2 of antibody 4E12

SEQ ID NO: 40 is a nucleotide sequence from a V_{H} CDR3 of antibody 4E12

SEQ ID NO: 41 is an amino acid sequence from a V_{L} CDR1 of antibody 4E12

SEQ ID NO: 42 is an amino acid sequence from a V_{L} CDR2 of antibody 4E12

SEQ ID NO: 43 is an amino acid sequence from a V_{L} CDR3 of antibody 4E12

SEQ ID NO: 44 is an amino acid sequence from a V_{H} CDR1 of antibody 4E12

SEQ ID NO: 45 is an amino acid sequence from a V_{H} CDR2 of antibody 4E12

SEQ ID NO: 46 is an amino acid sequence from a V_{H} CDR3 of antibody 4E12

### DETAILED DESCRIPTION

### General

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or groups of compositions of matter.

Those skilled in the art will appreciate that the present disclosure is susceptible to variations and modifications other than those specifically described. It is to be understood that the disclosure includes all such variations and modifications. The disclosure also includes all of the steps, features, compositions and compounds referred to or indicated in this specification, individually or collectively, and any and all combinations or any two or more of said steps or features.

The present disclosure is not to be limited in scope by the specific examples described herein, which are intended for the purpose of exemplification only. Functionally-equivalent products, compositions and methods are clearly within the scope of the present disclosure.

Any example of the present disclosure herein shall be taken to apply mutatis mutandis to any other example of the disclosure unless specifically stated otherwise.

Any example of the present disclosure in relation to treatment or prevention of a NAFLD shall be taken to apply mutatis mutandis to inhibiting or preventing an innate immune response (e.g., an innate immune response in the digestive system and/or a systemic innate immune response) in a subject suffering from a NAFLD.

Unless specifically defined otherwise, all technical and scientific terms used herein shall be taken to have the same meaning as commonly understood by one of ordinary skill in the art (for example, in cell culture, molecular genetics, immunology, immunohistochemistry, protein chemistry, and biochemistry).

Unless otherwise indicated, the recombinant protein, cell culture, and immunological techniques utilized in the present disclosure are standard procedures, well known to those skilled in the art. Such techniques are described and explained throughout the literature in sources such as, J. Perbal, A Practical Guide to Molecular Cloning, John Wiley and Sons (1984), J. Sambrook et al. Molecular Cloning: A Laboratory Manual, Cold Spring Harbor Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbor Laboratory, (1988), and J. E. Coligan et al. (editors) Current Protocols in Immunology, John Wiley & Sons (including all updates until present).

The description and definitions of variable regions and parts thereof, immunoglobulins, antibodies and fragments thereof herein may be further clarified by the discussion in Kabat Sequences of Proteins of Immunological Interest, National Institutes of Health, Bethesda, Md., 1987 and 1991, Bork et al., J Mol. Biol. 242, 309-320, 1994, Chothia and Lesk J. Mol Biol. 196:901-917, 1987, Chothia et al. Nature 342, 877-883, 1989 and/or or Al-Lazikani et al., J Mol Biol 273, 927-948, 1997.

Any discussion of a protein or antibody herein will be understood to include any variants of the protein or antibody produced during manufacturing and/or storage. For example, during manufacturing or storage an antibody can be deamidated (e.g., at an asparagine or a glutamine residue) and/or have altered glycosylation and/or have a glutamine residue converted to pyroglutamine and/or have a N-terminal or C-terminal residue removed or “clipped” and/or have part or all of a signal sequence incompletely processed and, as a consequence, remain at the terminus of the antibody. It is understood that a composition comprising a particular amino acid sequence may be a heterogeneous mixture of the stated or encoded sequence and/or variants of that stated or encoded sequence.

The term “and/or”, e.g., “X and/or Y” shall be understood to mean either “X and Y” or “X or Y” and shall be taken to provide explicit support for both meanings or for either meaning. Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

As used herein the term “derived from” shall be taken to indicate that a specified integer may be obtained from a particular source albeit not necessarily directly from that source.

### Selected Definitions

VEGF-B is known to exist in two major isoforms, referred to as VEGF-B₁₈₆ and VEGF-B₁₆₇. For the purposes of nomenclature only and not limitation exemplary sequences of human VEGF-B₁₈₆ is set out in NCBI Reference Sequence: NP_003368.1, in NCBI protein accession numbers NP_003368, P49765 and AAL79001 and in SEQ ID NO: 1. In the context of the present disclosure, the sequence of VEGF-B₁₈₆ can lack the 21 amino acid N-terminal signal sequence (e.g., as set out at amino acids 1 to 21 of SEQ ID NO: 1. For the purposes of nomenclature only and not limitation exemplary sequences of human VEGF-B₁₆₇ is set out in NCBI Reference Sequence: NP_001230662.1, in NCBI protein accession numbers AAL79000 and AAB06274 and in SEQ ID NO: 2. In the context of the present disclosure, the sequence of VEGF-B₁₆₇ can lack the 21 amino acid N-terminal signal sequence (e.g., as set out at amino acids 1 to 21 of SEQ ID NO: 2. Additional sequence of VEGF-B can be determined using sequences provided herein and/or in publically available databases and/or determined using standard techniques (e.g., as described in Ausubel et al., (editors), Current Protocols in Molecular Biology, Greene Pub. Associates and Wiley-Interscience (1988, including all updates until present) or Sambrook et al., Molecular Cloning: A Laboratory Manual, Cold Spring Harbor Laboratory Press (1989)). Reference to human VEGF-B may be abbreviated to hVEGF-B. In one example, reference herein to VEGF-B is to VEGF-B₁₆₇ isoform.

Reference herein to VEGF-B also encompasses the VEGF-B₁₀₋₁₀₈ peptide as described in WO2006/012688.

As used herein, “nonalcoholic fatty liver disease” or “NAFLD” refers to a condition in which fat is deposited in the liver (hepatic steatosis), with or without inflammation and fibrosis (i.e., hepatic fibrosis), in the absence of excessive alcohol use. This term encompasses steatosis, NASH and cirrhosis.

As used herein, a “subject with NAFLD” refers to a subject that has been diagnosed with NAFLD. In some examples, NAFLD is suspected during a routine checkup, monitoring of metabolic syndrome and obesity, or monitoring for possible side effects of drugs (e.g., cholesterol lowering agents or steroids). In some examples, liver enzymes such ASAT and ALAT are high. In some examples, a subject is diagnosed following abdominal or thoracic imaging, liver ultrasound, or magnetic resonance imaging. In some examples, other conditions such as excess alcohol consumption, hepatitis C, and Wilson's disease have been ruled out prior to an NAFLD diagnosis. In some examples, a subject has been diagnosed following a liver biopsy.

As used herein, “steatosis” and “non-alcoholic steatosis” are used interchangeably, and include mild, moderate, and severe steatosis, without inflammation or fibrosis, in the absence of excessive alcohol use.

As used here, the terms “hepatic” and “liver” are used interchangeably.

As used herein, a “subject with steatosis” and a “subject with non-alcoholic steatosis” are used interchangeably, and refer to a subject that has been diagnosed with steatosis. In some example, steatosis is diagnosed by a method described herein for NAFLD in general.

As used herein, “nonalcoholic steatohepatitis” or “NASH” refers to NAFLD in which there is inflammation and/or fibrosis in the liver (i.e., hepatic fibrosis). Exemplary methods of determining the stage of NASH are described, for example, in Kleiner et al, 2005, Hepatology, 41(6): 1313-1321, and Brunt et al, 2007, Modern Pathol, 20: S40-S48.

As used herein, a “subject with NASH” refers to a subject that has been diagnosed with NASH. In some examples, NASH is diagnosed by a method described above for NAFLD in general. In some examples, advanced fibrosis is diagnosed in a patient with NAFLD, for example, according to Gambino R, et.al. Annals of Medicine 2011; 43(8):617-49.

As used herein, the term “NASH-derived cirrhosis” or a “subject with NASH-derived cirrhosis” refers to a subject with cirrhosis that is caused by NASH, i.e., the NASH has progressed to cirrhosis.

As used herein, the term “NASH-associated cirrhosis” or “NASH-related cirrhosis” or a “subject with NASH-associated cirrhosis” refers to a subject that is diagnosed with cirrhosis and NASH, however the cirrhosis is not necessarily caused by the NASH.

As used herein, the term “NASH-associated hepatic fibrosis” or “NASH-related hepatic fibrosis” or a “subject with NASH-associated hepatic fibrosis” refers to a subject that is diagnosed with hepatic fibrosis and NASH, however the hepatic fibrosis is not necessarily caused by the NASH.

As used herein, the term “NASH-derived hepatocellular carcinoma” or a “subject with NASH-derived hepatocellular carcinoma” refers to a subject with hepatocellular carcinoma that is caused by NASH, i.e., the NASH has progressed to hepatocellular carcinoma.

As used herein, the term “NASH-associated hepatocellular carcinoma” or “NASH-related hepatocellular carcinoma” or a “subject with NASH-associated hepatocellular carcinoma” refers to a subject that is diagnosed with hepatocellular carcinoma and NASH, however the hepatocellular carcinoma is not necessarily caused by the NASH.

As used herein, “overweight” with reference to a subject refers to a subject with a BMI of 25 to <30.

As used herein, “obese” with reference to a subject refers to a subject with a BMI of 30 or greater.

As used herein, a “subject at risk of developing NAFLD” refers to a subject with one or more NAFLD comorbidities, such as obesity, abdominal obesity, metabolic syndrome, cardiovascular disease, and diabetes.

As used herein, a “subject at risk of developing steatosis” refers to a subject that has not been diagnosed as having steatosis, but who has one or more NAFLD comorbidities, such as obesity, abdominal obesity, metabolic syndrome, cardiovascular disease, and diabetes.

As used herein, a “subject at risk of developing NASH” refers to a subject with steatosis who continues to have one or more NAFLD comorbidities, such as obesity, abdominal obesity, metabolic syndrome, cardiovascular disease, and diabetes.

The term “recombinant” shall be understood to mean the product of artificial genetic recombination. Accordingly, in the context of a recombinant protein comprising an antibody variable region, this term does not encompass an antibody naturally-occurring within a subject's body that is the product of natural recombination that occurs during B cell maturation. However, if such an antibody is isolated, it is to be considered an isolated protein comprising an antibody variable region. Similarly, if nucleic acid encoding the protein is isolated and expressed using recombinant means, the resulting protein is a recombinant protein comprising an antibody variable region. A recombinant protein also encompasses a protein expressed by artificial recombinant means when it is within a cell, tissue or subject, e.g., in which it is expressed.

The term “protein” shall be taken to include a single polypeptide chain, i.e., a series of contiguous amino acids linked by peptide bonds or a series of polypeptide chains covalently or non-covalently linked to one another (i.e., a polypeptide complex). For example, the series of polypeptide chains can be covalently linked using a suitable chemical or a disulphide bond. Examples of non-covalent bonds include hydrogen bonds, ionic bonds, Van der Waals forces, and hydrophobic interactions.

The term “polypeptide” or “polypeptide chain” will be understood from the foregoing paragraph to mean a series of contiguous amino acids linked by peptide bonds.

The skilled artisan will be aware that an “antibody” is generally considered to be a protein that comprises a variable region made up of a plurality of polypeptide chains, e.g., a polypeptide comprising a light chain variable region (V_{L}) and a polypeptide comprising a heavy chain variable region (V_{H}). An antibody also generally comprises constant domains, some of which can be arranged into a constant region, which includes a constant fragment or fragment crystallizable (Fc), in the case of a heavy chain. A V_{H} and a V_{L} interact to form a Fv comprising an antigen binding region that is capable of specifically binding to one or a few closely related antigens. Generally, a light chain from mammals is either a λ light chain or a λ light chain and a heavy chain from mammals is α, δ, ε, γ, or μ. Antibodies can be of any type (e.g., IgG, IgE, IgM, IgD, IgA, and IgY), class (e.g., IgG₁, IgG₂, IgG₃, IgG₄, IgA₁ and IgA₂) or subclass. The term “antibody” also encompasses humanized antibodies, primatized antibodies, human antibodies, synhumanized antibodies and chimeric antibodies.

The terms “full-length antibody,” “intact antibody” or “whole antibody” are used interchangeably to refer to an antibody in its substantially intact form, as opposed to an antigen binding fragment of an antibody. Specifically, whole antibodies include those with heavy and light chains including an Fc region. The constant domains may be wild-type sequence constant domains (e.g., human wild-type sequence constant domains) or amino acid sequence variants thereof.

As used herein, “variable region” refers to the portions of the light and/or heavy chains of an antibody as defined herein that is capable of specifically binding to an antigen and includes amino acid sequences of complementarity determining regions (CDRs); i.e., CDR1, CDR2, and CDR3, and framework regions (FRs). Exemplary variable regions comprise three or four FRs (e.g., FR1, FR2, FR3 and optionally FR4) together with three CDRs. In the case of a protein derived from an IgNAR, the protein may lack a CDR2. V_{H} refers to the variable region of the heavy chain. V_{L} refers to the variable region of the light chain.

As used herein, the term “complementarity determining regions” (syn. CDRs; i.e., CDR1, CDR2, and CDR3) refers to the amino acid residues of an antibody variable domain the presence of which are necessary for antigen binding. Each variable domain typically has three CDR regions identified as CDR1, CDR2 and CDR3. The amino acid positions assigned to CDRs and FRs can be defined according to Kabat Sequences of Proteins of Immunological Interest, National Institutes of Health, Bethesda, Md., 1987 and 1991 or other numbering systems in the performance of this disclosure, e.g., the canonical numbering system of Chothia and Lesk J. Mol Biol. 196: 901-917, 1987; Chothia et al. Nature 342, 877-883, 1989; and/or Al-Lazikani et al., J Mol Biol 273: 927-948, 1997; the IMGT numbering system of Lefranc et al., Devel. And Compar. Immunol., 27: 55-77, 2003; or the AHO numbering system of Honnegher and Plukthun J. Mol. Biol., 309: 657-670, 2001.

“Framework regions” (FRs) are those variable domain residues other than the CDR residues.

As used herein, the term “Fv” shall be taken to mean any protein, whether comprised of multiple polypeptides or a single polypeptide, in which a V_{L} and a V_{H} associate and form a complex having an antigen binding site, i.e., capable of specifically binding to an antigen. The V_{H} and the V_{L} which form the antigen binding site can be in a single polypeptide chain or in different polypeptide chains. Furthermore, an Fv of the disclosure (as well as any protein of the disclosure) may have multiple antigen binding sites which may or may not bind the same antigen. This term shall be understood to encompass fragments directly derived from an antibody as well as proteins corresponding to such a fragment produced using recombinant means. In some examples, the V_{H} is not linked to a heavy chain constant domain (C_{H}) 1 and/or the V_{L} is not linked to a light chain constant domain (C_{L}). Exemplary Fv containing polypeptides or proteins include a Fab fragment, a Fab′ fragment, a F(ab′) fragment, a scFv, a diabody, a triabody, a tetrabody or higher order complex, or any of the foregoing linked to a constant region or domain thereof, e.g., C_{H}2 or C_{H}3 domain, e.g., a minibody. A “Fab fragment” consists of a monovalent antigen-binding fragment of an antibody, and can be produced by digestion of a whole antibody with the enzyme papain, to yield a fragment consisting of an intact light chain and a portion of a heavy chain or can be produced using recombinant means. A “Fab′ fragment” of an antibody can be obtained by treating a whole antibody with pepsin, followed by reduction, to yield a molecule consisting of an intact light chain and a portion of a heavy chain comprising a V_{H} and a single constant domain. Two Fab′ fragments are obtained per antibody treated in this manner. A Fab′ fragment can also be produced by recombinant means. A “F(ab′)2 fragment” of an antibody consists of a dimer of two Fab′ fragments held together by two disulfide bonds, and is obtained by treating a whole antibody molecule with the enzyme pepsin, without subsequent reduction. A “Fab₂” fragment is a recombinant fragment comprising two Fab fragments linked using, for example a leucine zipper or a C_{H}3 domain. A “single chain Fv” or “scFv” is a recombinant molecule containing the variable region fragment (Fv) of an antibody in which the variable region of the light chain and the variable region of the heavy chain are covalently linked by a suitable, flexible polypeptide linker.

As used herein, the term “binds” in reference to the interaction of a protein or an antigen binding site thereof with an antigen means that the interaction is dependent upon the presence of a particular structure (e.g., an antigenic determinant or epitope) on the antigen. For example, an antibody recognizes and binds to a specific protein structure rather than to proteins generally. If an antibody binds to epitope “A”, the presence of a molecule containing epitope “A” (or free, unlabeled “A”), in a reaction containing labeled “A” and the protein, will reduce the amount of labeled “A” bound to the antibody.

As used herein, the term “specifically binds” or “binds specifically” shall be taken to mean that a protein of the disclosure reacts or associates more frequently, more rapidly, with greater duration and/or with greater affinity with a particular antigen or cell expressing same than it does with alternative antigens or cells. For example, a protein binds to VEGF-B with materially greater affinity (e.g., 20 fold or 40 fold or 60 fold or 80 fold to 100 fold or 150 fold or 200 fold) than it does to other growth factor (e.g., VEGF-A) or to antigens commonly recognized by polyreactive natural antibodies (i.e., by naturally occurring antibodies known to bind a variety of antigens naturally found in humans). Generally, but not necessarily, reference to binding means specific binding, and each term shall be understood to provide explicit support for the other term.

As used herein, the term “neutralize” shall be taken to mean that a protein is capable of blocking, reducing or preventing VEGF-B-signaling in a cell through the VEGF-R1. Methods for determining neutralization are known in the art and/or described herein.

As used herein, the term “specifically inhibits VEGF-B signaling” will be understood to mean that the compound inhibits VEGF-B signaling and does not significantly or detectably inhibit signaling by one or more other VEGF proteins, e.g., VEGF-A, VEGF-B, VEGF-C, VEGF-D and/or PIGF.

As used herein, the term “does not significantly inhibit” shall be understood to mean that the level of inhibition of signaling by a VEGF protein other than VEGF-B (e.g., signalling by VEGF-A, VEGF-B, VEGF-C, VEGF-D and/or PIGF) in the presence of a compound described herein is not statistically significantly lower than in the absence of the compound described herein (e.g., in a control assay which may be conducted in the presence of an isotype control antibody).

As used herein, the term “does not detectably inhibit” shall be understood to mean that a compound as described herein inhibits signalling of a VEGF protein other than VEGF-B (e.g., signalling by VEGF-A, VEGF-B, VEGF-C, VEGF-D and/or PIGF) by no more than 10% or 8% or 6% or 5% or 4% or 3% or 2% or 1% of the level of signalling detected in the absence of the compound described herein (e.g., in a control assay which may be conducted in the presence of an isotype control antibody).

As used herein, the terms “preventing”, “prevent” or “prevention” include administering a compound of the disclosure to thereby stop or hinder the development of at least one symptom of a condition.

As used herein, the terms “treating”, “treat” or “treatment” include administering a protein described herein to thereby reduce or eliminate at least one symptom of a specified disease or condition or to slow progression of the disease or condition.

As used herein, the term “subject” shall be taken to mean any animal including humans, for example a mammal. Exemplary subjects include but are not limited to humans and non-human primates. For example, the subject is a human.

### Treatment of NAFLD

The present disclosure provides a method for treating or preventing a NAFLD in a subject, the method comprising administering to the subject an inhibitor of VEGF-B signaling.

In some examples, the methods include determining whether a subject has NAFLD, and selecting the subject if they do have NAFLD, then administering the compound that inhibits VEGF-B as described herein. Determining whether a subject has NAFLD can include reviewing their medical history, or ordering or performing such tests as are necessary to establish a diagnosis. Most individuals with NAFLD are asymptomatic; the condition is usually discovered incidentally as a result of abnormal liver function tests or hepatomegaly, e.g., noted in an unrelated medical condition. Elevated liver biochemistry is found in 50% of patients with simple steatosis (see, e.g., Sleisenger, Sleisenger and Fordtran's Gastrointestinal and Liver Disease. Philadelphia: W.B. Saunders Company (2006)). In general, the diagnosis begins with the presence of elevations in liver tests that are included in routine blood test panels, such as alanine aminotransferase (ALAT) or aspartate aminotransferase (ASAT). Even modest, subclinical increases in hepatic fat accumulation have been shown to be an early component in the progressive pathogenesis of metabolic syndrome (see, e.g., Almeda-Valdés et al., Ann. Hepatol. 8 Suppl 1:518-24 (2009); Polyzos et al., Curr Mol Med. 9(3):299-314 (2009); Byrne et al., Clin. Sci. (Lond). 116(7):539-64 (2009)).

Imaging studies are often obtained during evaluation process. Ultrasonography reveals a “bright” liver with increased echogenicity. Thus, medical imaging can aid in diagnosis of NAFLD; fatty livers have lower density than spleen on computed tomography (CT) and fat appears bright in T1-weighted magnetic resonance images (MRIs).

Making a differential diagnosis of Nonalcoholic Steatohepatitis (NASH), as opposed to simple fatty liver, is done using a liver biopsy. For a liver biopsy, a needle is inserted through the skin to remove a small piece of the liver. NASH is diagnosed when examination of the tissue with a microscope shows fat along with inflammation and damage to liver cells. If the tissue shows fat without inflammation and damage, simple NAFLD is diagnosed. Thus, histological diagnosis by liver biopsy is sought when assessment of severity is indicated.

In one example, the subject suffers from steatosis.

In one example, the subject suffers from NASH.

In one example, the subject suffers from cirrhosis. In one example, the cirrhosis is NASH-derived cirrhosis. In another example, the cirrhosis is NASH-associated cirrhosis.

In one example, a method of the disclosure prevents or slows progression of the NAFLD, e.g., from steatosis to NASH or from NASH to cirrhosis.

In one example, the subject is obese. For example, the subject has a BMI of 30 or greater. Accordingly, the present disclosure provides a method for treating NAFLD in an obese subject, the method comprising administering to the subject a compound that inhibits VEGF-B.

In one example, the subject suffers from metabolic syndrome. For example, the subject has diabetes mellitus, impaired glucose tolerance, impaired fasting glucose or insulin resistance, and two of the following:
Blood pressure: ≥140/90 mmHg;
Dyslipidemia: triglycerides (TG): ≥1.695 mmol/L and high-density lipoprotein cholesterol (HDL-C)≤0.9 mmol/L (male), ≤1.0 mmol/L (female);
Central obesity: waist:hip ratio >0.90 (male); >0.85 (female), or body mass index >30 kg/m²;
Microalbuminuria: urinary albumin excretion ratio ≥20 μg/min or albumin:creatinine ratio ≥30 mg/g.
In one example, the subject suffers from diabetes. For example, a subject suffering from diabetes has a clinically accepted marker of diabetes, such as:
Fasting plasma glucose of greater than or equal to 7 mmol/L or 126 mg/dl;
Casual plasma glucose (taken at any time of the day) of greater than or equal to 11.1 mmol/L or 200 mg/dl with the symptoms of diabetes.
Oral glucose tolerance test (OGTT) value of greater than or equal to 11.1 mmol/L or 200 mg/dl measured at a two-hour interval. The OGTT is given over a two or three-hour time span.
In one example, the subject suffers from type 2 diabetes.

### VEGF-B Signaling Inhibitors

### Proteins Comprising Antibody Variable Regions

An exemplary VEGF-B signaling inhibitor comprises an antibody variable region, e.g., is an antibody or an antibody fragment that binds to VEGF-B and neutralizes VEGF-B signaling.

In one example, the antibody variable region binds specifically to VEGF-B.

Suitable antibodies and proteins comprising variable regions thereof are known in the art.

For example, anti-VEGF-B antibodies and fragments thereof are described in WO2006/012688.

In one example, the anti-VEGF-B antibody or fragment thereof is an antibody that competitively inhibits the binding of 2H10 to VEGF-B or an antigen binding fragment thereof. In one example, the anti-VEGF-B antibody or fragment thereof is antibody 2H10 or a chimeric, CDR grafted or humanized version thereof or an antigen binding fragment thereof. In this regard, antibody 2H10 comprises a V_{H} comprising a sequence set forth in SEQ ID NO: 3 and a V_{L} comprising a sequence set forth in SEQ ID NO: 4. Exemplary chimeric and humanized versions of this antibody are described in WO2006/012688.

In one example, the anti-VEGF-B antibody or fragment thereof comprises a V_{H} comprising a sequence set forth in SEQ ID NO: 5 and a V_{L} comprising a sequence set forth in SEQ ID NO: 6.

In one example, the anti-VEGF-B antibody or fragment thereof is an antibody that competitively inhibits the binding of 4E12 to VEGF-B or an antigen binding fragment thereof. In one example, the anti-VEGF-B antibody or fragment thereof is antibody 4E12 or a chimeric, CDR grafted or humanized version thereof or an antigen binding fragment thereof. In this regard, antibody 4E12 comprises a V_{H} comprising a sequence set forth in SEQ ID NO: 7 and a V_{L} comprising a sequence set forth in SEQ ID NO: 8.

In one example, the compound is a protein comprising a humanized variable region of antibody 4E12. For example, the protein comprises a variable region comprising the complementarity determining regions (CDRs) of the V_{H} and/or the V_{L} of antibody 4E12. For example, the protein comprises:
(i) a V_{H} comprising:
   (a) a CDR1 comprising a sequence set forth in amino acids 25-34 of SEQ ID NO: 7;
   (b) a CDR2 comprising a sequence set forth in amino acids 49-65 of SEQ ID NO: 7; and
   (c) a CDR3 comprising a sequence set forth in amino acids 98-105 of SEQ ID NO: 7; and/or
(ii) a V_{L} comprising:
   (a) a CDR1 comprising a sequence set forth in amino acids 24-34 of SEQ ID NO: 8;
   (b) a CDR2 comprising a sequence set forth in amino acids 50-56 of SEQ ID NO: 8; and
   (c) a CDR3 comprising a sequence set forth in amino acids 89-97 of SEQ ID NO: 8.

In one example, the anti-VEGF-B antibody or fragment thereof is an antibody that competitively inhibits the binding of 2F5 to VEGF-B or an antigen binding fragment thereof. In one example, the anti-VEGF-B antibody or fragment thereof is antibody 2F5 or a chimeric, CDR grafted or humanized version thereof or an antigen binding fragment thereof. In this regard, antibody 2E5 comprises a V_{H} comprising a sequence set forth in SEQ ID NO: 9 and a V_{L} comprising a sequence set forth in SEQ ID NO: 10.

In one example, the compound is a protein comprising a humanized variable region of antibody 2F5. For example, the protein comprises a variable region comprising the complementarity determining regions (CDRs) of the V_{H} and/or the V_{L} of antibody 2F5. For example, the protein comprises:
(i) a V_{H} comprising:
   (a) a CDR1 comprising a sequence set forth in amino acids 25-34 of SEQ ID NO: 9;
   (b) a CDR2 comprising a sequence set forth in amino acids 49-65 of SEQ ID NO: 9; and
   (c) a CDR3 comprising a sequence set forth in amino acids 98-107 of SEQ ID NO: 9; and/or
(ii) a V_{L} comprising:
   (a) a CDR1 comprising a sequence set forth in amino acids 24-34 of SEQ ID NO: 10;
   (b) a CDR2 comprising a sequence set forth in amino acids 50-56 of SEQ ID NO: 10; and
   (c) a CDR3 comprising a sequence set forth in amino acids 89-96 of SEQ ID NO: 10.

In another example, an antibody or protein comprising a variable region thereof is produced using a standard method, e.g., as is known in the art or briefly described herein.

### Immunization-Based Methods

To generate antibodies, VEGF-B or an epitope bearing fragment or portion thereof or a modified form thereof or nucleic acid encoding same (an “immunogen”), optionally formulated with any suitable or desired adjuvant and/or pharmaceutically acceptable carrier, is administered to a subject (for example, a non-human animal subject, such as, a mouse, a rat, a chicken etc.) in the form of an injectable composition. Exemplary non-human animals are mammals, such as murine animals (e.g., rats or mice). Injection may be intranasal, intramuscular, sub-cutaneous, intravenous, intradermal, intraperitoneal, or by other known route. Optionally, the immunogen is administered numerous times. Means for preparing and characterizing antibodies are known in the art (See, e.g., Antibodies: A Laboratory Manual, Cold Spring Harbor Laboratory, 1988). Methods for producing anti-VEGF-B antibodies in mice are described in WO2006/012688.

The production of polyclonal antibodies may be monitored by sampling blood of the immunized animal at various points following immunization. A second, booster injection, may be given, if required to achieve a desired antibody titer. The process of boosting and subjecting is repeated until a suitable titer is achieved. When a desired level of immunogenicity is obtained, the immunized animal is bled and the serum isolated and stored, and/or the animal is used to generate monoclonal antibodies (mAbs).

Monoclonal antibodies are exemplary antibodies contemplated by the present disclosure. Generally, production of monoclonal antibodies involves, immunizing a subject (e.g., a rodent, e.g., mouse or rat) with the immunogen under conditions sufficient to stimulate antibody producing cells. In some examples, a mouse genetically-engineered to express human antibodies and not express murine antibodies proteins, is immunized to produce an antibody (e.g., as described in PCT/US2007/008231 and/or Lonberg et al., Nature 368 (1994): 856-859). Following immunization, antibody producing somatic cells (e.g., B lymphocytes) are fused with immortal cells, e.g., immortal myeloma cells. Various methods for producing such fused cells (hybridomas) are known in the art and described, for example, in Kohler and Milstein, Nature 256, 495-497, 1975. The hybridoma cells can then be cultured under conditions sufficient for antibody production.

The present disclosure contemplates other methods for producing antibodies, e.g., ABL-MYC technology (as described, for example in Largaespada et al, Curr. Top. Microbiol. Immunol, 166, 91-96. 1990).

### Library-Based Methods

The present disclosure also encompasses screening of libraries of antibodies or proteins comprising antigen binding domains thereof (e.g., comprising variable regions thereof) to identify a VEGF-B binding antibody or protein comprising a variable region thereof.

Examples of libraries contemplated by this disclosure include naïve libraries (from unchallenged subjects), immunized libraries (from subjects immunized with an antigen) or synthetic libraries. Nucleic acid encoding antibodies or regions thereof (e.g., variable regions) are cloned by conventional techniques (e.g., as disclosed in Sambrook and Russell, eds, Molecular Cloning: A Laboratory Manual, 3^{rd} Ed, vols. 1-3, Cold Spring Harbor Laboratory Press, 2001) and used to encode and display proteins using a method known in the art. Other techniques for producing libraries of proteins are described in, for example in U.S. Pat. No. 6,300,064 (e.g., a HuCAL library of Morphosys AG); U.S. Pat. Nos. 5,885,793; 6,204,023; 6,291,158; or U.S. Pat. No. 6,248,516.

The proteins according to the disclosure may be soluble secreted proteins or may be presented as a fusion protein on the surface of a cell, or particle (e.g., a phage or other virus, a ribosome or a spore). Various display library formats are known in the art. For example, the library is an in vitro display library (e.g., a ribosome display library, a covalent display library or a mRNA display library, e.g., as described in U.S. Pat. No. 7,270,969). In yet another example, the display library is a phage display library wherein proteins comprising antigen binding domains of antibodies are expressed on phage, e.g., as described in U.S. Pat. Nos. 6,300,064; 5,885,793; 6,204,023; 6,291,158; or U.S. Pat. No. 6,248,516. Other phage display methods are known in the art and are contemplated by the present disclosure. Similarly, methods of cell display are contemplated by the disclosure, e.g., bacterial display libraries, e.g., as described in U.S. Pat. No. 5,516,637; yeast display libraries, e.g., as described in U.S. Pat. No. 6,423,538 or a mammalian display library.

Methods for screening display libraries are known in the art. In one example, a display library of the present disclosure is screened using affinity purification, e.g., as described in Scopes (In: Protein purification: principles and practice, Third Edition, Springer Verlag, 1994). Methods of affinity purification typically involve contacting proteins comprising antigen binding domains displayed by the library with a target antigen (e.g., VEGF-B) and, following washing, eluting those domains that remain bound to the antigen.

Any variable regions or scFvs identified by screening are readily modified into a complete antibody, if desired. Exemplary methods for modifying or reformatting variable regions or scFvs into a complete antibody are described, for example, in Jones et al., J Immunol Methods. 354:85-90, 2010; or Jostock et al., J Immunol Methods, 289: 65-80, 2004. Alternatively, or additionally, standard cloning methods are used, e.g., as described in Ausubel et al (In: Current Protocols in Molecular Biology. Wiley Interscience, ISBN 047 150338, 1987), and/or (Sambrook et al (In: Molecular Cloning: Molecular Cloning: A Laboratory Manual, Cold Spring Harbor Laboratories, New York, Third Edition 2001).

### Deimmunized, Chimeric, Humanized, Synhumanized, Primatized and Human Proteins

The proteins of the present disclosure may be a humanized protein.

The term “humanized protein” shall be understood to refer to a protein comprising a human-like variable region, which includes CDRs from an antibody from a non-human species (e.g., mouse or rat or non-human primate) grafted onto or inserted into FRs from a human antibody (this type of antibody is also referred to a “CDR-grafted antibody”). Humanized proteins also include proteins in which one or more residues of the human protein are modified by one or more amino acid substitutions and/or one or more FR residues of the human protein are replaced by corresponding non-human residues. Humanized proteins may also comprise residues which are found in neither the human antibody or in the non-human antibody. Any additional regions of the protein (e.g., Fc region) are generally human. Humanization can be performed using a method known in the art, e.g., U.S. Pat. Nos. 5,225,539, 6,054,297, 7,566,771 or U.S. Pat. No. 5,585,089. The term “humanized protein” also encompasses a super-humanized protein, e.g., as described in U.S. Pat. No. 7,732,578.

The proteins of the present disclosure may be human proteins. The term “human protein” as used herein refers to proteins having variable and, optionally, constant antibody regions found in humans, e.g. in the human germline or somatic cells or from libraries produced using such regions. The “human” antibodies can include amino acid residues not encoded by human sequences, e.g. mutations introduced by random or site directed mutations in vitro (in particular mutations which involve conservative substitutions or mutations in a small number of residues of the protein, e.g. in 1, 2, 3, 4 or 5 of the residues of the protein). These “human antibodies” do not necessarily need to be generated as a result of an immune response of a human, rather, they can be generated using recombinant means (e.g., screening a phage display library) and/or by a transgenic animal (e.g., a mouse) comprising nucleic acid encoding human antibody constant and/or variable regions and/or using guided selection (e.g., as described in or U.S. Pat. No. 5,565,332). This term also encompasses affinity matured forms of such antibodies. For the purposes of the present disclosure, a human protein will also be considered to include a protein comprising FRs from a human antibody or FRs comprising sequences from a consensus sequence of human FRs and in which one or more of the CDRs are random or semi-random, e.g., as described in U.S. Pat. No. 6,300,064 and/or U.S. Pat. No. 6,248,516.

The proteins of the present disclosure may be synhumanized proteins. The term “synhumanized protein” refers to a protein prepared by a method described in WO2007/019620. A synhumanized protein includes a variable region of an antibody, wherein the variable region comprises FRs from a New World primate antibody variable region and CDRs from a non-New World primate antibody variable region. For example, a synhumanized protein includes a variable region of an antibody, wherein the variable region comprises FRs from a New World primate antibody variable region and CDRs from a mouse or rat antibody.

The proteins of the present disclosure may be primatized proteins. A “primatized protein” comprises variable region(s) from an antibody generated following immunization of a non-human primate (e.g., a cynomolgus macaque). Optionally, the variable regions of the non-human primate antibody are linked to human constant regions to produce a primatized antibody. Exemplary methods for producing primatized antibodies are described in U.S. Pat. No. 6,113,898.

In one example a protein of the disclosure is a chimeric protein. The term “chimeric proteins” refers to proteins in which an antigen binding domain is from a particular species (e.g., murine, such as mouse or rat) or belonging to a particular antibody class or subclass, while the remainder of the protein is from a protein derived from another species (such as, for example, human or non-human primate) or belonging to another antibody class or subclass. In one example, a chimeric protein is a chimeric antibody comprising a V_{H} and/or a V_{L} from a non-human antibody (e.g., a murine antibody) and the remaining regions of the antibody are from a human antibody. The production of such chimeric proteins is known in the art, and may be achieved by standard means (as described, e.g., in U.S. Pat. Nos. 6,331,415; 5,807,715; 4,816,567 and 4,816,397).

The present disclosure also contemplates a deimmunized protein, e.g., as described in WO2000/34317 and WO2004/108158. De-immunized antibodies and proteins have one or more epitopes, e.g., B cell epitopes or T cell epitopes removed (i.e., mutated) to thereby reduce the likelihood that a subject will raise an immune response against the antibody or protein.

### Other Proteins Comprising Antibody Variable Regions

The present disclosure also contemplates other proteins comprising a variable region or antigen binding domain of an antibody, such as:
(22) a single-domain antibody, which is a single polypeptide chain comprising all or a portion of the V_{H} or a V_{L} of an antibody (see, e.g., U.S. Pat. No. 6,248,516);
(ii) diabodies, triabodies and tetrabodies, e.g., as described in U.S. Pat. No. 5,844,094 and/or US2008152586;
(iii) scFvs, e.g., as described in U.S. Pat. No. 5,260,203;
(iv) minibodies, e.g., as described in U.S. Pat. No. 5,837,821;
(v) “key and hole” bispecific proteins as described in U.S. Pat. No. 5,731,168;
(vi) heteroconjugate proteins, e.g., as described in U.S. Pat. No. 4,676,980;
(vii) heteroconjugate proteins produced using a chemical cross-linker, e.g., as described in U.S. Pat. No. 4,676,980;
(viii) Fab′-SH fragments, e.g., as described in Shalaby et al, J. Exp. Med., 175: 217-225, 1992; or
(ix) Fab₃ (e.g., as described in EP19930302894).

### Constant Domain Fusions

The present disclosure encompasses a protein comprising a variable region of an antibody and a constant region or Fc or a domain thereof, e.g., C_{H}2 and/or C_{H}3 domain. Suitable constant regions and/or domains will be apparent to the skilled artisan and/or the sequences of such polypeptides are readily available from publicly available databases. Kabat et al also provide description of some suitable constant regions/domains.

Constant regions and/or domains thereof are useful for providing biological activities such as, dimerization, extended serum half-life e.g., by binding to FcRn (neonatal Fc Receptor), antigen dependent cell cytotoxicity (ADCC), complement dependent cytotoxicity (CDC, antigen dependent cell phagocytosis (ADCP).

The present disclosure also contemplates proteins comprising mutant constant regions or domains, e.g., as described in U.S. Pat. Nos. 7,217,797; 7,217,798; or US20090041770 (having increased half-life) or US2005037000 (increased ADCC).

### Stabilized Proteins

Neutralizing proteins of the present disclosure can comprise an IgG4 constant region or a stabilized IgG4 constant region. The term “stabilized IgG4 constant region” will be understood to mean an IgG4 constant region that has been modified to reduce Fab arm exchange or the propensity to undergo Fab arm exchange or formation of a half-antibody or a propensity to form a half antibody. “Fab arm exchange” refers to a type of protein modification for human IgG4, in which an IgG4 heavy chain and attached light chain (half-molecule) is swapped for a heavy-light chain pair from another IgG4 molecule. Thus, IgG4 molecules may acquire two distinct Fab arms recognizing two distinct antigens (resulting in bispecific molecules). Fab arm exchange occurs naturally in vivo and can be induced in vitro by purified blood cells or reducing agents such as reduced glutathione. A “half antibody” forms when an IgG4 antibody dissociates to form two molecules each containing a single heavy chain and a single light chain.

In one example, a stabilized IgG4 constant region comprises a proline at position 241 of the hinge region according to the system of Kabat (Kabat et al., Sequences of Proteins of Immunological Interest Washington DC United States Department of Health and Human Services, 1987 and/or 1991). This position corresponds to position 228 of the hinge region according to the EU numbering system (Kabat et al., Sequences of Proteins of Immunological Interest Washington DC United States Department of Health and Human Services, 2001 and Edelman et al., Proc. Natl. Acad. USA, 63, 78-85, 1969). In human IgG4, this residue is generally a serine. Following substitution of the serine for proline, the IgG4 hinge region comprises a sequence CPPC. In this regard, the skilled person will be aware that the “hinge region” is a proline-rich portion of an antibody heavy chain constant region that links the Fc and Fab regions that confers mobility on the two Fab arms of an antibody. The hinge region includes cysteine residues which are involved in inter-heavy chain disulfide bonds. It is generally defined as stretching from Glu226 to Pro243 of human IgG1 according to the numbering system of Kabat. Hinge regions of other IgG isotypes may be aligned with the IgG1 sequence by placing the first and last cysteine residues forming inter-heavy chain disulphide (S—S) bonds in the same positions (see for example WO2010/080538).

### Additional Protein-Based VEGF-B Signaling Inhibitors

Other proteins that may interfere with the productive interaction of VEGF-B with its receptor include mutant VEGF-B proteins.

In one example, the inhibitor is a soluble protein comprising one or more domains of a VEGF-R1 that bind to VEGF-B (and, e.g., do not substantially bind to VEGF-A). In one example, the soluble protein additionally comprises a constant region of an antibody, such as an IgG1 antibody. For example, the soluble protein additionally comprises a Fc region and, optionally a hinge region of an antibody, e.g., an IgG1 antibody.

In one example, the protein inhibitor is an antibody mimetic, e.g., a protein scaffold comprising variable regions that bind to a target protein in a manner analogous to an antibody. A description of exemplary antibody mimetics follows.

### Immunoglobulins and Immunoglobulin Fragments

An example of a compound of the present disclosure is a protein comprising a variable region of an immunoglobulin, such as a T cell receptor or a heavy chain immunoglobulin (e.g., an IgNAR, a camelid antibody).

### Heavy Chain Immunoglobulins

Heavy chain immunoglobulins differ structurally from many other forms of immunoglobulin (e.g., antibodies) in so far as they comprise a heavy chain, but do not comprise a light chain. Accordingly, these immunoglobulins are also referred to as “heavy chain only antibodies”. Heavy chain immunoglobulins are found in, for example, camelids and cartilaginous fish (also called IgNAR).

The variable regions present in naturally occurring heavy chain immunoglobulins are generally referred to as “V_{HH} domains” in camelid Ig and V-NAR in IgNAR, in order to distinguish them from the heavy chain variable regions that are present in conventional 4-chain antibodies (which are referred to as “V_{H} domains”) and from the light chain variable regions that are present in conventional 4-chain antibodies (which are referred to as “V_{L} domains”).

Heavy chain immunoglobulins do not require the presence of light chains to bind with high affinity and with high specificity to a relevant antigen. This means that single domain binding fragments can be derived from heavy chain immunoglobulins, which are easy to express and are generally stable and soluble.

A general description of heavy chain immunoglobulins from camelids and the variable regions thereof and methods for their production and/or isolation and/or use is found inter alia in the following references WO94/04678, WO97/49805 and WO 97/49805.

A general description of heavy chain immunoglobulins from cartilaginous fish and the variable regions thereof and methods for their production and/or isolation and/or use is found inter alia in WO2005/118629.

### V-Like Proteins

An example of a compound of the disclosure is a T-cell receptor. T cell receptors have two V-domains that combine into a structure similar to the Fv module of an antibody. Novotny et al., Proc Natl Acad Sci USA 88: 8646-8650, 1991 describes how the two V-domains of the T-cell receptor (termed alpha and beta) can be fused and expressed as a single chain polypeptide and, further, how to alter surface residues to reduce the hydrophobicity directly analogous to an antibody scFv. Other publications describing production of single-chain T-cell receptors or multimeric T cell receptors comprising two V-alpha and V-beta domains include WO1999/045110 or WO2011/107595.

Other non-antibody proteins comprising antigen binding domains include proteins with V-like domains, which are generally monomeric. Examples of proteins comprising such V-like domains include CTLA-4, CD28 and ICOS. Further disclosure of proteins comprising such V-like domains is included in WO1999/045110.

Adnectins

In one example, a compound of the disclosure is an adnectin. Adnectins are based on the tenth fibronectin type III (¹⁰Fn3) domain of human fibronectin in which the loop regions are altered to confer antigen binding. For example, three loops at one end of the β-sandwich of the ¹⁰Fn3 domain can be engineered to enable an Adnectin to specifically recognize an antigen. For further details see US20080139791 or WO2005/056764.

Anticalins

In a further example, a compound of the disclosure is an anticalin. Anticalins are derived from lipocalins, which are a family of extracellular proteins which transport small hydrophobic molecules such as steroids, bilins, retinoids and lipids. Lipocalins have a rigid β-sheet secondary structure with a plurality of loops at the open end of the conical structure which can be engineered to bind to an antigen. Such engineered lipocalins are known as anticalins. For further description of anticalins see U.S. Pat. No. 7,250,297B1 or US20070224633.

Affibodies

In a further example, a compound of the disclosure is an affibody. An affibody is a scaffold derived from the Z domain (antigen binding domain) of Protein A of Staphylococcus aureus which can be engineered to bind to antigen. The Z domain consists of a three-helical bundle of approximately 58 amino acids. Libraries have been generated by randomization of surface residues. For further details see EP1641818.

Avimers

In a further example, a compound of the disclosure is an Avimer. Avimers are multidomain proteins derived from the A-domain scaffold family. The native domains of approximately 35 amino acids adopt a defined disulphide bonded structure. Diversity is generated by shuffling of the natural variation exhibited by the family of A-domains. For further details see WO2002088171.

DARPins

In a further example, a compound of the disclosure is a Designed Ankyrin Repeat Protein (DARPin). DARPins are derived from Ankyrin which is a family of proteins that mediate attachment of integral membrane proteins to the cytoskeleton. A single ankyrin repeat is a 33 residue motif consisting of two α-helices and a β-turn. They can be engineered to bind different target antigens by randomizing residues in the first α-helix and a β-turn of each repeat. Their binding interface can be increased by increasing the number of modules (a method of affinity maturation). For further details see US20040132028.

### Methods for Producing Proteins

### Recombinant Expression

In the case of a recombinant protein, nucleic acid encoding same can be cloned into expression vectors, which are then transfected into host cells, such as E. coli cells, yeast cells, insect cells, or mammalian cells, such as simian COS cells, Chinese Hamster Ovary (CHO) cells, human embryonic kidney (HEK) cells, or myeloma cells that do not otherwise produce an antibody. Exemplary cells used for expressing a protein of the disclosure are CHO cells, myeloma cells or HEK cells. Molecular cloning techniques to achieve these ends are known in the art and described, for example in Ausubel et al., (editors), Current Protocols in Molecular Biology, Greene Pub. Associates and Wiley-Interscience (1988, including all updates until present) or Sambrook et al., Molecular Cloning: A Laboratory Manual, Cold Spring Harbor Laboratory Press (1989). A wide variety of cloning and in vitro amplification methods are suitable for the construction of recombinant nucleic acids. Methods of producing recombinant antibodies are also known in the art. See U.S. Pat. No. 4,816,567 or U.S. Pat. No. 5,530,101.

Following isolation, the nucleic acid is inserted operably linked to a promoter in an expression construct or expression vector for further cloning (amplification of the DNA) or for expression in a cell-free system or in cells.

As used herein, the term “promoter” is to be taken in its broadest context and includes the transcriptional regulatory sequences of a genomic gene, including the TATA box or initiator element, which is required for accurate transcription initiation, with or without additional regulatory elements (e.g., upstream activating sequences, transcription factor binding sites, enhancers and silencers) that alter expression of a nucleic acid, e.g., in response to a developmental and/or external stimulus, or in a tissue specific manner. In the present context, the term “promoter” is also used to describe a recombinant, synthetic or fusion nucleic acid, or derivative which confers, activates or enhances the expression of a nucleic acid to which it is operably linked. Exemplary promoters can contain additional copies of one or more specific regulatory elements to further enhance expression and/or alter the spatial expression and/or temporal expression of said nucleic acid.

As used herein, the term “operably linked to” means positioning a promoter relative to a nucleic acid such that expression of the nucleic acid is controlled by the promoter.

Many vectors for expression in cells are available. The vector components generally include, but are not limited to, one or more of the following: a signal sequence, a sequence encoding an antibody (e.g., derived from the information provided herein), an enhancer element, a promoter, and a transcription termination sequence. The skilled artisan will be aware of suitable sequences for expression of an antibody. Exemplary signal sequences include prokaryotic secretion signals (e.g., pelB, alkaline phosphatase, penicillinase, Ipp, or heat-stable enterotoxin II), yeast secretion signals (e.g., invertase leader, a factor leader, or acid phosphatase leader) or mammalian secretion signals (e.g., herpes simplex gD signal).

Exemplary promoters active in mammalian cells include cytomegalovirus immediate early promoter (CMV-IE), human elongation factor 1-α promoter (EF1), small nuclear RNA promoters (U1a and U1b), α-myosin heavy chain promoter, Simian virus 40 promoter (SV40), Rous sarcoma virus promoter (RSV), Adenovirus major late promoter, j-actin promoter; hybrid regulatory element comprising a CMV enhancer/β-actin promoter or an immunoglobulin promoter or active fragment thereof. Examples of useful mammalian host cell lines are monkey kidney CV1 line transformed by SV40 (COS-7, ATCC CRL 1651); human embryonic kidney line (293 or 293 cells subcloned for growth in suspension culture; baby hamster kidney cells (BHK, ATCC CCL 10); or Chinese hamster ovary cells (CHO).

Typical promoters suitable for expression in yeast cells such as for example a yeast cell selected from the group comprising Pichia pastoris, Saccharomyces cerevisiae and S. pombe, include, but are not limited to, the ADH1 promoter, the GAL1 promoter, the GAL4 promoter, the CUP1 promoter, the PHO5 promoter, the nmt promoter, the RPR1 promoter, or the TEF1 promoter.

Means for introducing the isolated nucleic acid or expression construct comprising same into a cell for expression are known to those skilled in the art. The technique used for a given cell depends on the known successful techniques. Means for introducing recombinant DNA into cells include microinjection, transfection mediated by DEAE-dextran, transfection mediated by liposomes such as by using lipofectamine (Gibco, MD, USA) and/or cellfectin (Gibco, MD, USA), PEG-mediated DNA uptake, electroporation and microparticle bombardment such as by using DNA-coated tungsten or gold particles (Agracetus Inc., WI, USA) amongst others.

The host cells used to produce the antibody may be cultured in a variety of media, depending on the cell type used. Commercially available media such as Ham's F10 (Sigma), Minimal Essential Medium ((MEM), (Sigma), RPMI-1640 (Sigma), and Dulbecco's Modified Eagle's Medium ((DMEM), Sigma) are suitable for culturing mammalian cells. Media for culturing other cell types discussed herein are known in the art.

### Protein Purification

Following production/expression, a protein of the disclosure is purified using a method known in the art. Such purification provides the protein of the disclosure substantially free of nonspecific protein, acids, lipids, carbohydrates, and the like. In one example, the protein will be in a preparation wherein more than about 90% (e.g. 95%, 98% or 99%) of the protein in the preparation is a protein of the disclosure.

Standard methods of peptide purification are employed to obtain an isolated protein of the disclosure, including but not limited to various high-pressure (or performance) liquid chromatography (HPLC) and non-HPLC polypeptide isolation protocols, such as size exclusion chromatography, ion exchange chromatography, hydrophobic interaction chromatography, mixed mode chromatography, phase separation methods, electrophoretic separations, precipitation methods, salting in/out methods, immunochromatography, and/or other methods.

In one example, affinity purification is useful for isolating a fusion protein comprising a label. Methods for isolating a protein using affinity chromatography are known in the art and described, for example, in Scopes (In: Protein purification: principles and practice, Third Edition, Springer Verlag, 1994). For example, an antibody or compound that binds to the label (in the case of a polyhistidine tag this may be, for example, nickel-NTA) is immobilized on a solid support. A sample comprising a protein is then contacted to the immobilized antibody or compound for a time and under conditions sufficient for binding to occur. Following washing to remove any unbound or non-specifically bound protein, the protein is eluted.

In the case of a protein comprising a Fc region of an antibody, protein A or protein G or modified forms thereof can be used for affinity purification. Protein A is useful for isolating purified proteins comprising a human γ1, γ2, or γ4 heavy chain Fc region. Protein G is recommended for all mouse Fc isotypes and for human γ3.

### Nucleic Acid-Based VEGF-B Signaling Inhibitors

In one example of the disclosure, therapeutic and/or prophylactic methods as described herein according to any example of the disclosure involve reducing expression of VEGF-B. For example, such a method involves administering a compound that reduces transcription and/or translation of the nucleic acid. In one example, the compound is a nucleic acid, e.g., an antisense polynucleotide, a ribozyme, a PNA, an interfering RNA, a siRNA, a microRNA

### Antisense Nucleic Acids

The term “antisense nucleic acid” shall be taken to mean a DNA or RNA or derivative thereof (e.g., LNA or PNA), or combination thereof that is complementary to at least a portion of a specific mRNA molecule encoding a polypeptide as described herein in any example of the disclosure and capable of interfering with a post-transcriptional event such as mRNA translation. The use of antisense methods is known in the art (see for example, Hartmann and Endres (editors), Manual of Antisense Methodology, Kluwer (1999)).

An antisense nucleic acid of the disclosure will hybridize to a target nucleic acid under physiological conditions. Antisense nucleic acids include sequences that correspond to structural genes or coding regions or to sequences that effect control over gene expression or splicing. For example, the antisense nucleic acid may correspond to the targeted coding region of a nucleic acid encoding VEGF-B, or the 5′-untranslated region (UTR) or the 3′-UTR or combination of these. It may be complementary in part to intron sequences, which may be spliced out during or after transcription, for example only to exon sequences of the target gene. The length of the antisense sequence should be at least 19 contiguous nucleotides, for example, at least 50 nucleotides, such as at least 100, 200, 500 or 1000 nucleotides of a nucleic acid encoding VEGF-B. The full-length sequence complementary to the entire gene transcript may be used. The length can be 100-2000 nucleotides. The degree of identity of the antisense sequence to the targeted transcript should be at least 90%, for example, 95-100%.

Exemplary antisense nucleic acids against VEGF-B are described, for example, in WO2003/105754.

### Catalytic Nucleic Acid

The term “catalytic nucleic acid” refers to a DNA molecule or DNA-containing molecule (also known in the art as a “deoxyribozyme” or “DNAzyme”) or a RNA or RNA-containing molecule (also known as a “ribozyme” or “RNAzyme”) which specifically recognizes a distinct substrate and catalyzes the chemical modification of this substrate. The nucleic acid bases in the catalytic nucleic acid can be bases A, C, G, T (and U for RNA).

Typically, the catalytic nucleic acid contains an antisense sequence for specific recognition of a target nucleic acid, and a nucleic acid cleaving enzymatic activity (also referred to herein as the “catalytic domain”). The types of ribozymes that are useful in this disclosure are a hammerhead ribozyme and a hairpin ribozyme.

### RNA Interference

RNA interference (RNAi) is useful for specifically inhibiting the production of a particular protein. Without being limited by theory, this technology relies on the presence of dsRNA molecules that contain a sequence that is essentially identical to the mRNA of the gene of interest or part thereof, in this case an mRNA encoding a VEGF-B. Conveniently, the dsRNA can be produced from a single promoter in a recombinant vector host cell, where the sense and anti-sense sequences are flanked by an unrelated sequence which enables the sense and anti-sense sequences to hybridize to form the dsRNA molecule with the unrelated sequence forming a loop structure. The design and production of suitable dsRNA molecules for the present disclosure is well within the capacity of a person skilled in the art, particularly considering WO99/32619, WO99/53050, WO99/49029, and WO01/34815.

The length of the sense and antisense sequences that hybridize should each be at least 19 contiguous nucleotides, such as at least 30 or 50 nucleotides, for example at least 100, 200, 500 or 1000 nucleotides. The full-length sequence corresponding to the entire gene transcript may be used. The lengths can be 100-2000 nucleotides. The degree of identity of the sense and antisense sequences to the targeted transcript should be at least 85%, for example, at least 90% such as, 95-100%.

Exemplary small interfering RNA (“siRNA”) molecules comprise a nucleotide sequence that is identical to about 19-21 contiguous nucleotides of the target mRNA. For example, the siRNA sequence commences with the dinucleotide AA, comprises a GC-content of about 30-70% (for example, 30-60%, such as 40-60% for example about 45%-55%), and does not have a high percentage identity to any nucleotide sequence other than the target in the genome of the mammal in which it is to be introduced, for example as determined by standard BLAST search. Exemplary siRNA that reduce expression of VEGF-B are commercially available from Santa Cruz Biotechnology or Novus Biologicals.

Short hairpin RNA (shRNA) that reduce expression of VEGF-B are also known in the art and commercially available from Santa Cruz Biotechnology.

### Screening Assays

Compounds that inhibit VEGF-B signaling can be identified using techniques known in the art, e.g., as described below. Similarly, amounts of VEGF-B signaling inhibitors suitable for use in a method described herein can be determined or estimated using techniques known in the art, e.g., as described below.

### Neutralization Assays

For compounds that bind to VEGF-B and inhibit signaling, a neutralization assay can be used.

In one example, a neutralization assay involves contacting VEGF-B with a compound in the presence or absence of detectably labeled soluble VEGF-R1 or contacting detectably labeled VEGF-B with a compound in the presence or absence of a cell expressing VEGF-R1 or a soluble VEGF-R1. The level of VEGF-B bound to the VEGF-R1 is then assessed. A reduced level of bound VEGF-B in the presence of the compound compared to in the absence of the compound indicates the compound inhibits VEGF-B binding to VEGF-R1 and, as a consequence VEGF-B signaling.

Another neutralization assay is described in WO2006/012688 and involves contacting a fragment of VEGF-R1 comprising the second Ig-like domain immobilized on a solid support with a subsaturating concentration of recombinant VEGF-B pre-incubated with a compound. Following washing to remove unbound protein, the immobilized protein is contacted with anti-VEGF-B antibody and the amount of bound antibody (indicative of immobilized VEGF-B) determined. A compound that reduces the level of bound antibody compared to the level in the absence of the compound is considered an inhibitor of VEGF-B signaling.

In another example, a compound that inhibits VEGF-B signaling is identified using a cell dependent on VEGF-B signaling for proliferation, e.g., a BaF3 cell modified as described in WO2006/012688 to express a chimeric receptor incorporating the intracellular domain of the human erythropoietin receptor and the extracellular domain of VEGF-R1. Cells are cultured in the presence of VEGF-B and in the presence or absence of a compound. Cell proliferation is then assessed using standard methods, e.g., colony formation assays, thymidine incorporation or uptake of another suitable marker of cell proliferation (e.g., a MTS dye reduction assay). A compound that reduces the level of proliferation in the presence of VEGF-B is considered an inhibitor of VEGF-B signaling.

Compounds can also be assessed for their ability to bind to VEGF-B using standard methods. Methods for assessing binding to a protein are known in the art, e.g., as described in Scopes (In: Protein purification: principles and practice, Third Edition, Springer Verlag, 1994). Such a method generally involves labeling the compound and contacting it with immobilized VEGF-B. Following washing to remove non-specific bound compound, the amount of label and, as a consequence, bound compound is detected. Of course, the compound can be immobilized and the VEGF-B labeled. Panning-type assays can also be used. Alternatively, or additionally, surface plasmon resonance assays can be used.

### Expression Assays

A compound that reduces or prevents expression of VEGF-B is identified by contacting a cell with the compound and determining the level of expression of the VEGF-B. Suitable methods for determining gene expression at the nucleic acid level are known in the art and include, for example, quantitative polymerase chain reaction (qPCR) or microarray assays. Suitable methods for determining expression at the protein level are also known in the art and include, for example, enzyme-linked immunosorbent assay (ELISA), fluorescence linked immunosorbent assay (FLISA), immunofluorescence or Western blotting.

### In Vivo Assays

Compounds described herein can be tested in an animal model of a NAFLD.

For example, mice (e.g., C57/BL6 mice) fed a high fat diet show similar metabolic features seen in human NASH with obesity, impaired glucose tolerance, insulin resistance, dyslipidemia and increased expression of regulators of lipogenesis and proinflammatory cytokines.

In another example, mice (e.g., C57/BL6 mice) fed a choline-deficient high fat diet show similar features of human NASH including obesity, impaired glucose tolerance, insulin resistance, immune cell infiltration and satellitosis. These mice can also go on to develop fibrosis and cirrhosis and in the long-term hepatocellular carcinomas.

Another suitable mouse model is induced by feeding mice a “Western diet” comprising high fats and high fructose levels. These mice exhibit obesity, insulin resistance, dyslipidemia, hyperglycemia and NAFLD.

Other suitable diet-based models include mice fed with a diet low in methionine.

There are also numerous genetic models of NAFLD/NASH, such as sterol regulatory element binding protein (SREBP)-1c-transgenic mice and phosphatase and tensin homologue deleted on chromosome 10 (PTEN)-null mice. In these models hepatic steatosis occurs first, followed subsequently by the development of steatohepatitis.

### Pharmaceutical Compositions and Methods of Treatment

A compound that inhibits VEGF-B signaling (syn. Active ingredient) is useful for parenteral, topical, oral, or local administration, aerosol administration, or transdermal administration, for prophylactic or for therapeutic treatment. In one example, the compound is administered parenterally, such as subcutaneously or intravenously.

Formulation of a compound to be administered will vary according to the route of administration and formulation (e.g., solution, emulsion, capsule) selected. An appropriate pharmaceutical composition comprising compound to be administered can be prepared in a physiologically acceptable carrier. For solutions or emulsions, suitable carriers include, for example, aqueous or alcoholic/aqueous solutions, emulsions or suspensions, including saline and buffered media. Parenteral vehicles can include sodium chloride solution, Ringer's dextrose, dextrose and sodium chloride, lactated Ringer's or fixed oils. A variety of appropriate aqueous carriers are known to the skilled artisan, including water, buffered water, buffered saline, polyols (e.g., glycerol, propylene glycol, liquid polyethylene glycol), dextrose solution and glycine. Intravenous vehicles can include various additives, preservatives, or fluid, nutrient or electrolyte replenishers (See, generally, Remington's Pharmaceutical Science, 16^{th} Edition, Mack, Ed. 1980). The compositions can optionally contain pharmaceutically acceptable auxiliary substances as required to approximate physiological conditions such as pH adjusting and buffering agents and toxicity adjusting agents, for example, sodium acetate, sodium chloride, potassium chloride, calcium chloride and sodium lactate. The compound can be lyophilized for storage and reconstituted in a suitable carrier prior to use according to art-known lyophilization and reconstitution techniques.

The optimum concentration of the active ingredient(s) in the chosen medium can be determined empirically, according to procedures known to the skilled artisan, and will depend on the ultimate pharmaceutical formulation desired.

The dosage ranges for the administration of the compound of the disclosure are those large enough to produce the desired effect. For example, the composition comprises a therapeutically or prophylactically effective amount of the compound.

As used herein, the term “effective amount” shall be taken to mean a sufficient quantity of the compound to inhibit/reduce/prevent signaling of VEGF-B in a subject. The skilled artisan will be aware that such an amount will vary depending on, for example, the compound and/or the particular subject and/or the type and/or the severity of NAFLD being treated. Accordingly, this term is not to be construed to limit the disclosure to a specific quantity, e.g., weight or number of compounds.

As used herein, the term “therapeutically effective amount” shall be taken to mean a sufficient quantity of compound to reduce or inhibit one or more symptoms of a NAFLD or a complication thereof.

As used herein, the term “prophylactically effective amount” shall be taken to mean a sufficient quantity of compound to prevent or inhibit or delay the onset of one or more detectable symptoms of a NAFLD or a complication thereof.

In one example, the compound is administered in an amount effective to have one or more of the following effects:
Reduce or prevent lipid accumulation, e.g., neutral lipids in the liver of a subject, e.g., as assessed in a liver biopsy;
Reduce or prevent inflammation in the liver of the subject, e.g., by reducing the number of immune cells in the liver of the subject;
Reduce or prevent development of pathologic changes of NAFLD, such as, Mallory-Denk bodies or hepatocyte ballooning or inflammatory foci or satellitosis in the liver of a subject;
Reduce or prevent hepatic fibrosis and/or cirrhosis;
Reduce or prevent formation of hepatocellular carcinoma.

In one example, the compound is administered in an amount sufficient to reduce the level of lipid accumulation in the liver of a subject to the level seen in a population of subjects not suffering from a NAFLD.

The dosage should not be so large as to cause adverse side effects, such as hyper viscosity syndromes, pulmonary edema, congestive heart failure, and the like. Generally, the dosage will vary with the age, condition, sex and extent of the disease in the patient and can be determined by one of skill in the art. The dosage can be adjusted by the individual physician in the event of any complication.

Dosage can vary from about 0.1 mg/kg to about 300 mg/kg, e.g., from about 0.2 mg/kg to about 200 mg/kg, such as, from about 0.5 mg/kg to about 20 mg/kg, in one or more dose administrations daily, for one or several days.

In some examples, the compound is administered at an initial (or loading) dose which is higher than subsequent (maintenance doses). For example, the compound is administered at an initial dose of between about 1 mg/kg to about 30 mg/kg. The compound is then administered at a maintenance dose of between about 0.0001 mg/kg to about 1 mg/kg. The maintenance doses may be administered every 7-35 days, such as, every 14 or 21 or 28 days.

In some examples, a dose escalation regime is used, in which a compound is initially administered at a lower dose than used in subsequent doses. This dosage regime is useful in the case of subject's initially suffering adverse events

In the case of a subject that is not adequately responding to treatment, multiple doses in a week may be administered. Alternatively, or in addition, increasing doses may be administered.

A subject may be retreated with the compound, by being given more than one exposure or set of doses, such as at least about two exposures of the compound, for example, from about 2 to 60 exposures, and more particularly about 2 to 40 exposures, most particularly, about 2 to 20 exposures.

In one example, any retreatment may be given when signs or symptoms of disease return, e.g., when the microalbuminuria progresses.

In another example, any retreatment may be given at defined intervals. For example, subsequent exposures may be administered at various intervals, such as, for example, about 24-28 weeks or 48-56 weeks or longer. For example, such exposures are administered at intervals each of about 24-26 weeks or about 38-42 weeks, or about 50-54 weeks.

A method of the present disclosure may also include co-administration of the at least one compound according to the disclosure together with the administration of another therapeutically effective agent for the prevention or treatment of diabetes and/or obesity.

In one example, the compound(s) of the disclosure is used in combination with at least one additional known compound which is currently being used or is in development for preventing or treating diabetes. Examples of such known compounds include but are not limited to common anti-diabetic drugs such as sulphonylureas (e.g. glicazide, glipizide), metformin, glitazones (e.g. rosiglitazone, pioglitazone), sodium-glucose cotransporter-2 (SGLT2) inhibitors (e.g., canagliflozin, dapagliflozin, empagliflozin), prandial glucose releasing agents (e.g. repaglinide, nateglinide), acarbose and insulin (including all naturally-occurring, synthetic and modified forms of insulin, such as insulin of human, bovine or porcine origin; insulin suspended in, for example, isophane or zinc and derivatives such as insulin glulisine, insulin lispro, insulin lispro protamine, insulin glargine, insulin detemir or insulin aspart).

Additionally, the methods of the disclosure may also include co-administration of at least one other therapeutic agent for the treatment of another disease directly or indirectly related to NAFLD. Additional examples of agents that can be co-administered with the compound(s) according to the invention are compounds used to reduce cholesterol and triglycerides (e.g. fibrates (e.g., Gemfibrozil™) and HMG-CoA inhibitors such as Lovastatin™, Atorvastatin™ Fluvastatin™, Lescol™), Lipitor™, Mevacor™), Pravachol™, Pravastatin™, Simvastatin™ Zocor™, Cerivastatin™), etc); compounds that inhibit intestinal absorption of lipids (e.g., ezetiminde); nicotinic acid; farnesoid X receptor agonists (e.g., obeticholic acid, 6alpha-ethyl-chenodeoxycholic acid (6-ECDCA)) and Vitamin D.

As will be apparent from the foregoing, the present disclosure provides methods of concomitant therapeutic treatment of a subject, comprising administering to a subject in need thereof an effective amount of a first compound and a second compound, wherein said first compound is a compound of the disclosure (i.e., an inhibitor of VEGF-B signaling), and the second compound is for the prevention or treatment of diabetes or obesity.

As used herein, the term “concomitant” as in the phrase “concomitant therapeutic treatment” includes administering a first compound in the presence of a second compound. A concomitant therapeutic treatment method includes methods in which the first, second, third or additional compounds are co-administered. A concomitant therapeutic treatment method also includes methods in which the first or additional compounds are administered in the presence of a second or additional compounds, wherein the second or additional compounds, for example, may have been previously administered. A concomitant therapeutic treatment method may be executed step-wise by different actors. For example, one actor may administer to a subject a first compound and as a second actor may administer to the subject a second compound and the administering steps may be executed at the same time, or nearly the same time, or at distant times, so long as the first compound (and/or additional compounds) are after administration in the presence of the second compound (and/or additional compounds). The actor and the subject may be the same entity (e.g. a human).

In one example, the disclosure also provides a method for treating or preventing a NAFLD in a subject, the method comprising administering to the subject a first pharmaceutical composition comprising at least one compound of the disclosure and a second pharmaceutical composition comprising one or more additional compounds.

In one example, a method of the disclosure comprises administering an inhibitor of VEGF-B signaling to a subject suffering from NAFLD and receiving another treatment (e.g., for diabetes).

### Kits

Another example of the disclosure provides kits containing compounds useful for the treatment of NAFLD as described above.

In one example, the kit comprises (a) a container comprising a compound that inhibits VEGF-B signaling as described herein, optionally in a pharmaceutically acceptable carrier or diluent; and (b) a package insert with instructions for treating a NAFLD or complication thereof in a subject.

In accordance with this example of the disclosure, the package insert is on or associated with the container. Suitable containers include, for example, bottles, vials, syringes, etc. The containers may be formed from a variety of materials such as glass or plastic. The container holds or contains a composition that is effective for treating the NAFLD and may have a sterile access port (for example, the container may be an intravenous solution bag or a vial having a stopper pierceable by a hypodermic injection needle). At least one active agent in the composition is the compound that inhibits VEGF-B signaling. The label or package insert indicates that the composition is used for treating a subject eligible for treatment, e.g., one having or predisposed to a NAFLD, with specific guidance regarding dosing amounts and intervals of compound and any other medicament being provided. The kit may further comprise an additional container comprising a pharmaceutically acceptable diluent buffer, such as bacteriostatic water for injection (BWFI), phosphate-buffered saline, Ringer's solution, and/or dextrose solution. The kit may further include other materials desirable from a commercial and user standpoint, including other buffers, diluents, filters, needles, and syringes.

The kit optionally further comprises a container comprises a second medicament, wherein the compound that inhibits VEGF-B signaling is a first medicament, and which article further comprises instructions on the package insert for treating the subject with the second medicament, in an effective amount. The second medicament may be any of those set forth above.

The present disclosure includes the following non-limiting Examples.

### Example 1: Mice Deficient in VEGF-B are Resistant to the Development of Nonalcoholic Fatty Liver Disease (NAFLD) and Nonalcoholic Steatohepatitis (NASH)

Diabetic mice deficient in VEGF-B are protected from hepatic damage Five week old C57BL/6 wild-type (WT) and C57BL/6-Vegfb−/− mice were fed with high fat diet (60% calories from fat) for 30 weeks. Aged and sex matched C57BL/6 WT mice were fed a low fat control diet (normal chow, 10% calories from fat) for 30 weeks. Blood glucose was measured bi-weekly at the same time of the day after withdrawal of the food for 2 hours as a mean to stabilize the blood glucose levels. The tip of the tail was cut and a drop of blood measured with a glucose meter. At end-point serum aminotransferase (ALAT) levels were measured.

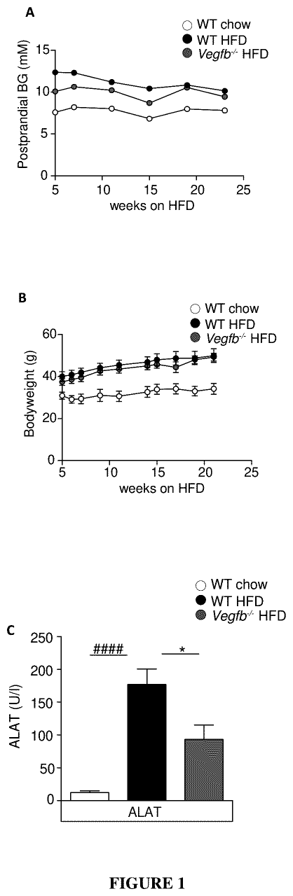

FIGS. 1A and 1B show high fat diet (HFD) fed mice exhibited elevated blood glucose levels and body weight compared to age-matched chow-fed mice (FIGS. 1A and 1B respectively),

FIG. 1C shows serum ALAT levels were increased by 15 fold in HFD-fed mice, whilst HFD-fed mice deficient in VEGF-B had decreased serum ALAT levels compared to age-matched HFD-fed WT mice.

These data demonstrate that ablation of Vegfb in HFD-fed mice decreases hepatic damage, without targeting hyperglycemia.

Deletion of Vegfb reduces hepatic lipid accumulation in HFD-fed mice Oil red O (ORO) analysis was performed on isolated livers from HFD-fed WT, HFD-fed Vegfb^{-/-} and chow-fed WT mice. Briefly, livers were dissected and flash frozen on dry ice and embedded in Tissue-Tek® (Sakura) directly on the mold of the cryostat. Cryosections (12 m) were immersed 5 min in ORO working solution (2.5 g oil red O (Sigma-Aldrich), dissolved in 400 ml 99% isopropanol, further diluted 6:10 in H₂O, filtered through a 22 μm filter (Corning)) and submerged for 3 sees in haematoxylin solution followed by short submerging in LiCO₃ and rinsed 10 min under running tap water before they were mounted. At least 10 frames per animal, stained for ORO and haematoxylin within each section were photographed with bright field microscopy (Axio Vision microscope, Carl Zeiss) at 20× magnification. The amount of lipid droplets was quantified using Axio Vision Run wizard program for liver ORO staining (pixel²/μm²).

Expression levels of fatty acid synthase (Fasn) were detected in isolated livers. Total RNA was extracted and purified from livers using the Rneasy Mini Kit (Qiagen) according to the manufacturer's instructions. First strand cDNA was synthesized from 0.5-1 μg total RNA using iScript cDNA Synthesis Kit (Bio-Rad). Real-Time quantitive PCR was performed using KAPA SYBR FAST qPCR Kit Master Mix (2×) Universal (KAPA Biosystems) in Rotor-Gene Q (Qiagen) Real-Time PCR thermal cycler according to the manufacturer's instructions. Expression levels were normalized to the expression of L19 and β-2 microglobulin.

Hepatic lipid droplet accumulation and structure were ameliorated in HFD-fed mice with reduced expression of VEGF-B. In particular, the lipid droplets were reduced in number and size in hepatic sections from Vegfb deficient HFD-fed mice, compared to WT HFD-fed mice.

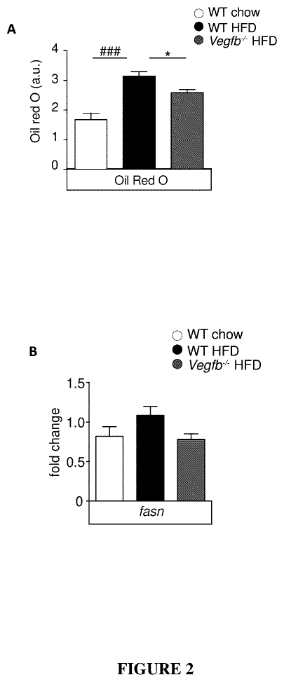

FIG. 2 shows that reducing the levels of VEGF-B in HFD-fed mice decreases the hepatic content of neutral lipids.

### Deletion of Vegfb in HFD-Fed Mice Prevents Development of Hepatic Steatosis

HFD-fed WT, HFD-fed Vegfb^{-/-} and chow-fed WT mice were used for analysis. Livers were dissected, fixed in 4% PFA for 24 h and subsequently processed for paraffin embedding using standard procedures and 6 μm sections were prepared. Briefly, antigen retrieval was performed using antigen retrieval solution pH6 (Dako #S2367) and heated at 98° C. for 10 minutes. Sections were incubated at 4° C. overnight with primary antibodies: guinea pig anti-adipophilin (Fitzgerald) or guinea pig anti-tip47 (Progen) antibodies. Before addition of appropriate fluorescently labeled secondary antibodies (Invitrogen, Alexa Fluor) samples were incubated with biotinylated donkey anti-guinea pig antibody (Jackson) for 1 hour at RT. At least 10 frames per animal stained for adipophilin or tip47 within each section were photographed with an Axio Vision microscope (Carl Zeiss) at 20× magnification. The amount of respective staining were quantified using Axio Vision Run wizard program for hepatic i) adipophilin staining (pixel²/μm²) or ii) tip47 staining (pixel²/μm²).

Expression of adipophilin (plin2a) in chow-fed WT, HFD-fed WT and HFD-fed Vegfb^{-/-} -mice was also determined using methods described above.

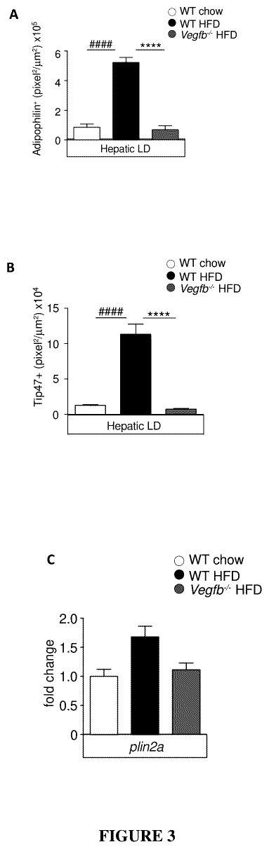

FIG. 3 shows that the lipid content in livers of HFD-fed Vegfb^{-/-} mice is reduced by 5-10 folds as measured by expression of PAT proteins, adipophilin (A, C) and mannose-6-phosphate receptor binding protein 1 (tip47; B) compared to HFD-fed WT mice. Decreasing VEGF-B expression preserves liver morphology with smaller lipid droplets in HFD-fed Vegfb^{-/-} mice compared to HFD-fed WT mice. These data indicate reducing levels of VEGF-B in HFD-fed mice prevents the development of hepatic steatosis and that the VEGF-B signaling pathway is a suitable target for treating non-alcoholic fatty liver disease.

### Deletion of Vegfb in HFD-Fed Mice Prevents Development of Hepatic Inflammation

HFD-fed WT, HFD-fed Vegfb^{-/-} and normal chow-fed WT mice were used for analysis. Livers were isolated and flash frozen on dry ice. Liver biopsies were embedded in Tissue-Tek® (Sakura) directly on the mold of the cryostat. After embedding, 12-am sections were prepared, post-fixated in ice-cold 4% PFA and thereafter immunostained for CD45 or F4/80. Briefly, sections were incubated at 4° C. for 12 h with primary rat anti-CD45 (BD bioscience) and rat anti-F4/80 (Serotec) antibodies. Appropriate fluorescently labeled secondary antibodies (Invitrogen, Alexa fluor) were applied and sections were further incubated for 1 h at RT after which they were prepared for microscopy. At least 10 frames per animal stained for CD45 or F4/80 within each section were photographed with an Axio Vision microscope (Carl Zeiss) at 20× magnification. The amount of each staining were quantified using Axio Vision Run wizard program for hepatic i) CD45 staining (pixel²/μm²) or ii) F4/80 staining (pixel²/μm²).

Expression of monocyte chemoattractant protein-1 (mcp1) in chow-fed WT, HFD-fed WT and HFD-fed Vegfb^{-/-} mice was also determined using methods described above.

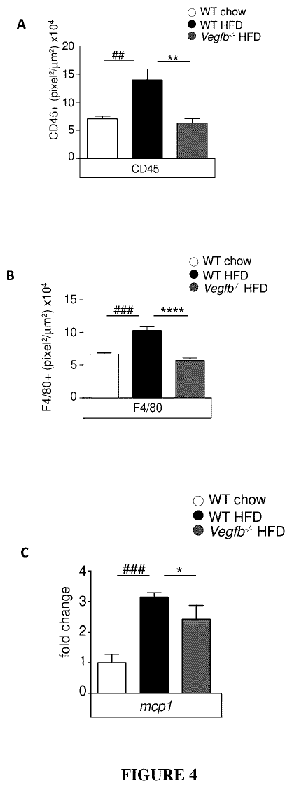

As shown in FIG. 4, the inflammatory cellular population in steatotic livers was increased 2-3 folds by HFD-feeding (FIGS. 4A & 4B). The increase in liver inflammation was verified by up-regulation of mcp1 in HFD-fed mice (FIG. 4C). Both hepatic steatosis and the number of inflammatory cells were decreased in HFD-fed mice with reduced expression of VEGF-B (FIGS. 4A-C).

### Deletion of Vegfb in HFD-Fed Mice Reduces NASH and NASH Associated Pathologies

HFD-fed WT, HFD-fed Vegfb^{-/-} and chow-fed WT mice were used for analysis. Livers were dissected, post-fixated in 4% PFA for 24 h and subsequently processed for paraffin embedding using standard procedures. After embedding, 6-μm sections were prepared and stained with Hematoxylin-Eosin (H&E) (Sigma) according to the manufacturer's instructions. At least 20 frames per animal stained for H&E within each section were photographed with bright field microscopy (Axio Vision microscope, Carl Zeiss) at 40× magnification and analysed for the presence of ballooned hepatocytes, MDB formation, inflammatory foci and satellitosis of H&E-stained sections. Scoring was based on the number of balloon cells, MDB formation/inflammatory foci or satellitosis in each frame of H&E-stained sections. For each animal, the average of all identified ballooned hepatocytes, MDBs, inflammatory foci and satellitosis in H&E stained liver sections was calculated. The NASH total score was calculated as the sum of the averages within each group.

As shown in Table 1, the livers of HFD-fed mice displayed ballooned hepatocytes and MDB formation, and to a lesser extent, immune cell infiltration and satellitosis. Reducing VEGF-B levels in HFD-fed mice decreased the appearance of these human NASH associated pathologies.

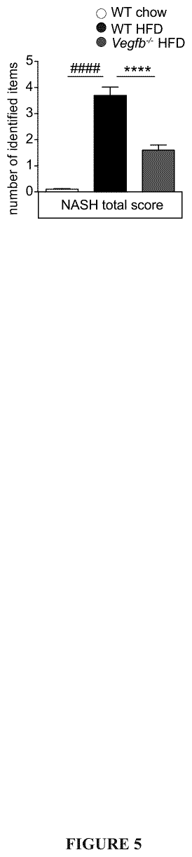

FIG. 5 shows that reducing VEGF-B levels in HFD-fed mice the total NASH score is reduced by more than 50%, compared to HFD-fed mice.

TABLE 1
Hepatic NASH scores in HFD-fed mice with genetic ablation of Vegfb

| | Item | | | |
|---|---|---|---|---|
| | Hepatocellular | | Inflammatory | Satelli- |
| Mice | ballooning | MDB | foci | tosis |
| WT chow (n = 5) | 0.1 ± 0.1 | 0.0 ± 0.0 | 0.0 ± 0.0 | 0.0 ± 0.0 |
| WT HFD (n = 9) | 2.4 ± 0.2^{####} | 0.7 ± 0.1^{####} | 0.4 ± 0.1 | 0.4 ± 0.1 |
| Vegfb^{-l-} HFD | 1.1 ± 0.2^{####} | 0.3 ± 0.1* | 0.1 ± 0.0 | 0.0 ± 0.0 |
| (n = 6) | | | | |

Results are expressed as mean s.e.m.
^{####}P < 0.0001 compared to chow-fed WT and
*P < 0.05 compared to HFD-fed WT mice.
Statistical analyses were performed using two-way ANOVA.

### Example 2: A Neutralizing Anti-VEGF-B Antibody Treats or Prevents Progression of Nonalcoholic Fatty Liver Disease (NAFLD) and Nonalcoholic Steatohepatitis (NASH)

### Antibody-Mediated Inhibition of VEGF-B Moderately Influences Hepatic Function in HFD-Fed Mice

Five week old C57BL/6 wild-type (WT) were fed with high fat diet (60% calories from fat) 30 weeks. Aged and sex matched C57BL/6 WT mice were fed a low fat control diet (normal chow, 10% calories from fat) for 30 weeks. Antibody treatment commenced at week 11 and mice were injected intraperitoneally twice weekly with 400 μg 2H10 (neutralizing anti-VEGF-B antibody) or isotype-matched control antibody for 20 weeks. Postprandial blood glucose levels of mice were monitored bi-weekly after removal of food for 2 hrs. Glucose measurements were performed on blood drawn from the tail vein using a Bayer Contour Glucose meter. End-point serum aminotransferase (ALAT) levels were measured.

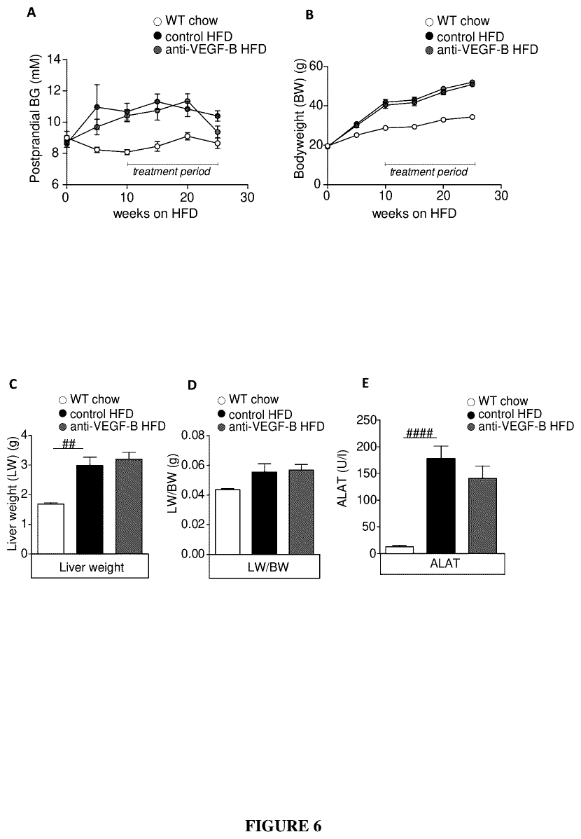

FIGS. 6A and B show blood glucose levels and body weight of HFD-fed mice treated therapeutically with antibody 2H10.

FIGS. 6C and D show liver weight and ratio of liver weight and body weight in HFD-fed mice treated therapeutically with antibody 2H10.

FIG. 6D shows serum analysis of ALAT levels in HFD-fed mice treated therapeutically with antibody 2H10.

These data suggest that reducing VEGF-B levels using anti-VEGF-B antibody treatment (2H10) can prevent the decrease in liver function due to HFD-feeding.

### Therapeutic Anti-VEGF-B Treatment (Using 2H10) Reduces Hepatic Lipid Accumulation in HFD-Fed Mice

Oil red O (ORO) analysis was performed on isolated livers from 30 week old chow-fed WT and HFD-fed mice treated with 2H10 or isotype-matched control antibody for 20 weeks. Livers were collected, flash frozen on dry ice and embedded in Tissue-Tek® (Sakura) directly on the mold of the cryostat. Cryosections (12 m) were immersed 5 min in ORO working solution (2.5 g ORO (Sigma-Aldrich), dissolved in 400 ml 99% isopropanol, further diluted 6:10 in H2O, filtered through a 22 μm filter (Corning)). Thereafter the sections were submerged for 3 secs in hematoxylin solution followed by submerging in LiCO₃ and rinsed for 10 min under running tap water before they were mounted. Stained sections were examined with bright field microscopy. At least 10 frames per animal stained for ORO and hematoxylin within each section were photographed with an Axio Vision microscope (Carl Zeiss) at 20× magnification. The amounts of lipid droplets were quantified using Axio Vision Run wizard program for liver ORO staining (pixel²/μm²).

Expression of fatty acid synthase (fasn) was also determined using methods described above.

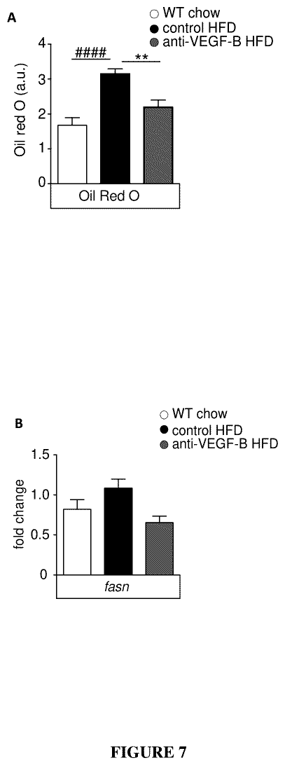

As shown in FIG. 7, the amount of ORO staining was reduced in HFD-fed mice receiving anti-VEGF-B antibody treatment. These data and analysis of the stained sections indicate that both numbers and size of lipid droplets were reduced in HFD-fed mice treated with anti-VEGF-B antibody and that administration of 2H10 in HFD-fed mice protects against hepatic accumulation of neutral lipids.

### Therapeutic Anti-VEGF-B Treatment (Using 2H10) Prevents Development of Hepatic Steatosis

Livers from 30 week old HFD-fed mice treated with 2H10 or isotype-matched control antibody for 20 weeks were collected, fixed in 4% PFA for 24 hours, embedded and 6 μm sections prepared for immunostaining. Briefly antigen retrieval was performed using Antigen retrieval solution pH6 (Dako #S2367) and heated at 98° C. for 10 min. Sections were incubated at 4° C. overnight with primary antibodies: guinea pig anti-adipophilin (Fitzgerald) or guinea pig anti-tip47 (Progen) antibodies. Before addition of appropriate fluorescently labeled secondary antibodies (Invitrogen, Alexa Fluor) samples were incubated with biotinylated donkey anti-guinea pig antibody (Jackson) for 1 hour at RT. At least 10 frames per animal stained for adipophilin or tip47 within each section were photographed with an Axio Vision microscope (Carl Zeiss) at 20× magnification. The amount of respective staining were quantified using Axio Vision Run wizard program for hepatic i) adipophilin staining (pixel²/μm²) or ii) tip47 staining (pixel²/μm²).

Expression of adipophilin (plin2a) was also determined using methods described above.

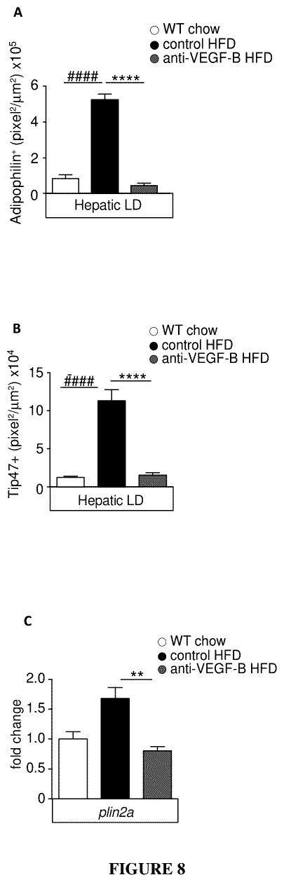

FIG. 8 shows anti-VEGF-B antibody treatment in HFD-fed mice, using 2H10, reduced expression of PAT proteins in steatotic liver. Anti-VEGF-B antibody treatment reduced hepatic lipid content to similar levels as those obtained in chow-fed WT and HFD-fed Vegfb^{-/-} mice. The impact on the development of hepatic steatosis was verified by hepatic expression analysis as 2H10 treatment in HFD-fed mice decreased mRNA transcript levels of plin2.

### Therapeutic Anti-VEGF-B Treatment (Using 2H10) Prevents Development of Hepatic Inflammation in HFD-Fed Mice

Livers from 30 week old HFD-fed mice treated with 2H10 or isotype-matched control antibody for 20 weeks were collected, and flash frozen on dry ice and embedded in Tissue-Tek® (Sakura) directly on the mold of the cryostat. After embedding, 12-μm sections were prepared, post-fixated in ice-cold 4% PFA for 10 minutes and thereafter immunostained for CD45 or F4/80. Briefly, sections were incubated at 4° C. for 12 h with primary rat anti-CD45 (BD bioscience) and rat anti-F4/80 (Serotec) antibodies. Appropriate fluorescently labeled secondary antibodies (Invitrogen, Alexa fluor) were applied and sections were further incubated for 1 h at RT after which they were prepared for microscopy. At least 10 frames per animal stained for CD45 or F4/80 within each section were photographed with an Axio Vision microscope (Carl Zeiss) at 20× magnification. The amount of respective staining were quantified using Axio Vision Run wizard program for hepatic i) CD45 staining (pixel²/μm²) or ii) F4/80 staining (pixel²/μm²).

Expression of monocyte chemoattractant protein-1 (mcp1) was also determined using methods described above.

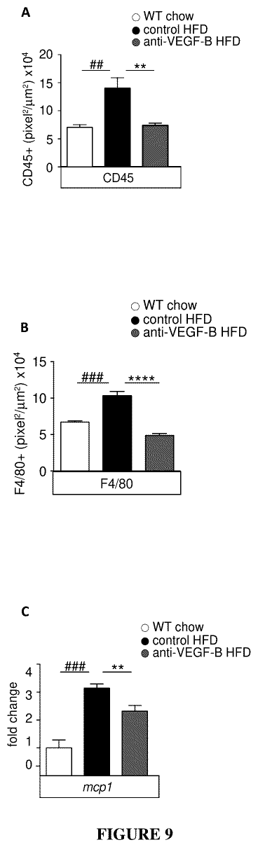

FIG. 9 shows decreased levels of CD45 (A) and F4/80 (B) levels in livers of HFD-fed mice treated therapeutically with anti-VEGF antibody. Treatment of HFD-fed mice with anti-VEGF-B antibody 2H10 decreased the number of hepatic inflammatory cells to similar levels as chow-fed WT and HFD-fed Vegfb^{-/-} mice. Expression levels of mcp1 were also decreased by anti-VEGF-B antibody treatment. Thus, these data indicate that therapeutic treatment with an anti-VEGF-B antibody prevents the development of the main pathologies in NASH.

### Therapeutic Anti-VEGF-B Treatment (Using 2H10) Reduces NASH and NASH Associated Pathologies

Livers from 30 week old HFD-fed mice treated with 2H10 or isotype-matched control antibody for 20 weeks were collected, fixed in 4% PFA for 24 hours, embedded and 6 μm sections prepared and stained with Hematoxylin-Eosin (H&E) (Sigma) according to the manufacturer instructions. At least 20 frames per animal stained for H&E within each section were photographed with bright field microscopy (Axio Vision microscope, Carl Zeiss) at 40× magnification. For each animal, the average of all identified ballooned hepatocytes, MDBs, inflammatory foci and satellitosis in H&E stained liver sections was calculated. The NASH total score was calculated as the sum of the averages within each group.

As shown in Table 2, the livers of HFD-fed mice displayed ballooned hepatocytes, MDB formation, and to a lesser extent, immune cell infiltration and satellitosis. Therapeutic treatment with an anti-VEGF-B antibody decreased the appearance of these human NASH associated pathologies.

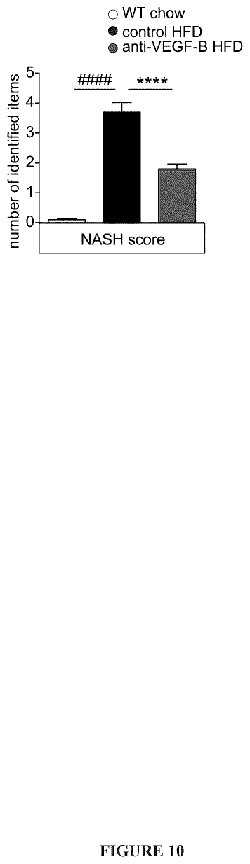

FIG. 10 shows that reducing VEGF-B levels in HFD-fed mice using 2H10 antibody treatment decreased the total NASH score by more than 50%.

TABLE 2
Hepatic NASH scores in HFD-fed mice treated with 2H10 or
control antibody

| | Item | | | |
|---|---|---|---|---|
| | Hepatocellular | | Inflammatory | Satelli- |
| Mice | ballooning | MDB | foci | tosis |
| WT chow (n = 5) | 0.1 ± 0.1 | 0.0 ± 0.0 | 0.0 ± 0.0 | 0.0 ± 0.0 |
| control HFD | 2.4 ± 0.2^{####} | 0.7 ± 0.1^{####} | 0.4 ± 0.1 | 0.4 ± 0.1 |
| (n = 9) | | | | |
| anti-VEGF-B | 1.2 ± 0.2^{####} | 0.3 ± 0.1* | 0.2 ± 0.0 | 0.1 ± 0.0 |
| HFD | | | | |
| (n = 9) | | | | |

Results are expressed as mean s.e.m.
^{####}P < 0.0001 compared to chow-fed WT animals and
*P < 0.05 compared to control treated HFD-fed mice. Statistical analyses were performed using two-way ANOVA.

### Example 3: Mice Deficient in VEGF-B on Short-Term Choline-Deficient High Fat (CD) Diet are Resistant to the Development of Nonalcoholic Fatty Liver Disease (NAFLD) and Nonalcoholic Steatohepatitis (NASH)

Deletion of Vegfb increases glucose and insulin sensitivity Five week old male C57BL/6 mice and aged matched Vegfb^{+/-} and Vegfb^{-/-} mice where fed a choline-deficient high fat (CD) diet (Research Diets; D05010402) for 5 months (CD5). Body weight (BW) and blood glucose (BG) levels were recorded during the trial. The food was removed for 2 h prior to BG recording. Glucose measurements were performed on blood drawn from the tail vein using a Bayer Contour Glucose meter. Intraperitoneal glucose tolerance tests (IPGTT) and intraperitoneal insulin tolerance tests (IPITT) were performed after 17 weeks on CD-diet on un-starved mice or on mice that had their food removed 2 h before the experiment. For the tolerance tests animals were injected intraperitoneally with 1 mg glucose per g BW (IPGTT) and with 0.75 mU insulin per g BW (IPITT).

Short-term CD lead to increased body weight gain, blood glucose levels, glucose intolerance and insulin resistance in C57BL/6 mice. Ablation of Vegfb in C57BL/6 mice on CD diet did not alter body weight or the development of hyperglycemia. However, reducing VEGF-B levels in C57BL/6 mice on CD diet by genetic means had some effect on glucose tolerance and insulin sensitivity, but did not target hyperglycaemia.

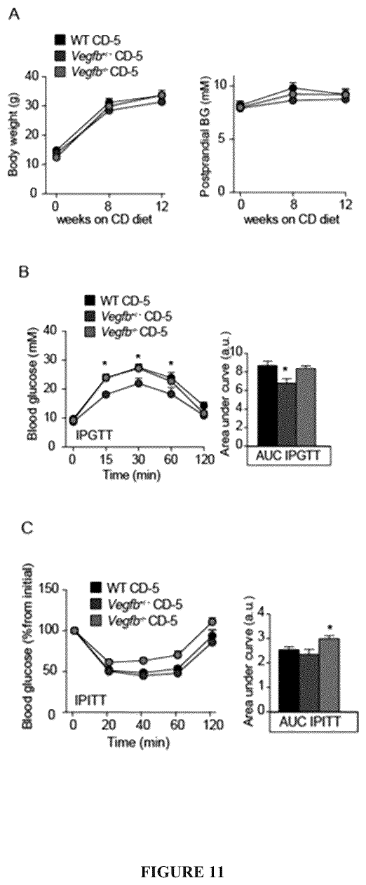

FIG. 11 shows that deletion of Vegfb did not impact on body weight (A) or postprandial blood glucose levels (A) but increased glucose and insulin sensitivity (B and C) in mice on short-term choline-deficient high fat (CD) diet.

Deletion of Vegfb Protects from Hepatic Damage

C57BL/6, Vegfb^{-/-} and Vegfb^{-/-} CD fed mice were used for analysis. Livers were dissected and weighed and total blood was removed by cardiac puncture. The blood was centrifuged at 14000 rpm, 4° C. for 10 minutes, serum was separated and frozen in aliquots at −80° C. Alanine aminotransferase (ALAT) serum levels were analyzed at the Swedish University of Agricultural Science in Uppsala, Sweden.

Short-term CD induced liver injury and serum ALAT levels were increased in WT mice, as compared to Vegfb^{+/-} and Vegfb^{-/-} mice.

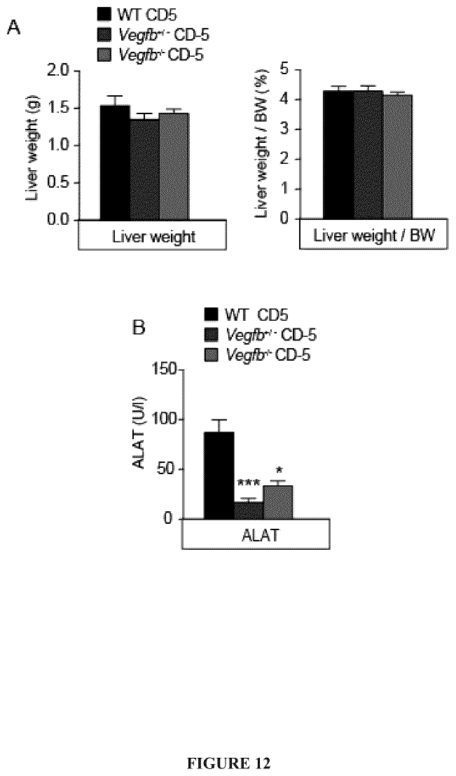

FIG. 12A shows that deletion of Vegfb in mice on short-term CD diet had no effect on liver weight or liver weight/body weight ratio.

FIG. 12B shows that serum ALAT levels were increased in CD-fed WT mice, whilst CD-fed mice deficient in VEGF-B had decreased serum ALAT levels compared to age-matched CD-fed WT mice.

These data demonstrate that ablation of Vegfb in CD-fed mice decreases hepatic damage, without targeting hyperglycemia.

### Deletion of Vegfb Reduces Hepatic Lipid Accumulation

C57BL/6, Vegfb^{-/-} and Vegfb^{+/-} CD fed mice were used for analysis. Livers were dissected and flash-frozen and total blood was removed by cardiac puncture. The blood was centrifuged at 14000 rpm, 4° C. for 10 minutes, serum separated and frozen in aliquots at −80° C.

Oil red O (ORO) analysis was performed on isolated livers. Briefly Liver biopsies were embedded in Tissue-Tek® (Sakura) directly on the mold of the cryostat. Cryosections (12 μm) were immersed 5 min in oil red O working solution (2.5 g oil red O (ORO; Sigma-Aldrich), dissolved in 400 ml 99% isopropanol, further diluted 6:10 in H₂O, filtered through a 22 μm filter (Corning). Thereafter the sections were submerged for 3 sec in hematoxylin solution followed by short submerging in LiCO₃ and rinsed for 10 min under running tap water before they were mounted. Stained sections were examined with bright field microscopy. At least 10 frames per animal stained for ORO and hematoxylin within each section were photographed with an Axio Vision microscope (Carl Zeiss) at 20× magnification. The amount of lipid droplets was quantified using Axio Vision Run wizard program for liver ORO staining (pixel²/μm²).

Commercially available kits were used for enzymatic determination of non-esterified fatty acids (NEFAs; Wako Chemicals), beta-hydroxybutyrate (Stanbio Laboratories) and triglycerides (Sigma-Aldrich).

Short-term CD led to the development of non-alcoholic fatty acid liver disease (NAFLD) in C57BL/6 mice. CD diet induced a dramatic increase in hepatic content of neutral lipids, as detected by ORO analysis, and a concomitant decrease in liver function (FIG. 12). Genetic ablation of Vegfb in C57BL/6 mice on CD diet decreased hepatic lipid accumulation. The lipid droplets were reduced both in number and size. Short-term CD lead to dyslipidemia and reducing VEGF-B levels decreased plasma levels of ketone bodies (KBs), NEFAs and triglycerides (TGs).

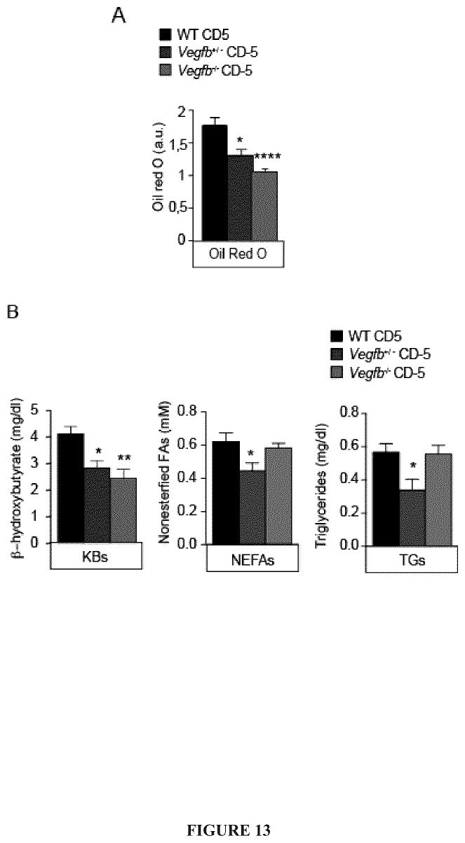

FIG. 13 shows that deletion of Vegfb reduced hepatic lipid accumulation in mice on short-term CD diet and targeted hepatic steatosis.

### Deletion of Vegfb Prevents the Development of Hepatic Inflammation

C57BL/6, Vegfb^{-/-} and Vegfb^{+/-} CD fed mice were used for analysis. Liver biopsies were embedded in Tissue-Tek® (Sakura) directly on the mold of the cryostat. After embedding, 12-μm sections were prepared, post-fixated in ice-cold 4% PFA and thereafter immunostained for CD45 or F4/80. Briefly, sections were incubated at 4° C. for 12 h with primary rat anti-CD45 (BD bioscience) and rat anti-F4/80 (Serotec) antibodies. Appropriate fluorescently labeled secondary antibodies (Invitrogen, Alexa fluor) were applied and sections were further incubated for 1 h at RT after which they were prepared for microscopy. At least 10 frames per animal stained for CD45 or F4/80 within each section were photographed with an Axio Vision microscope (Carl Zeiss) at 20× magnification. The amount of respective staining were quantified using Axio Vision Run wizard program for hepatic i) CD45 staining (pixel²/μm²) or ii) F4/80 staining (pixel²/μm²).

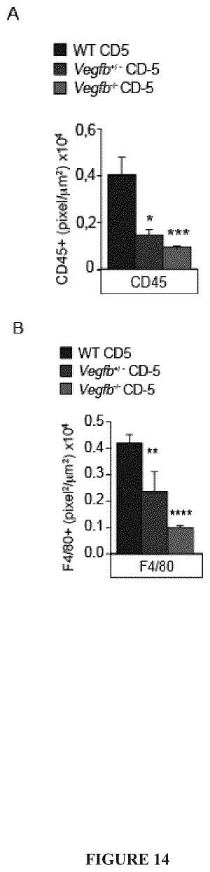

Short-term CD can be used to induce the full spectrum of non-alcoholic steatohepatitis disease (NASH) i.e., abnormal hepatic lipid accumulation and inflammation in C57BL/6 mice. Both hepatic steatosis (FIGS. 13 and 14) and the number of inflammatory cells were decreased in mice with homozygous or heterozygous ablation of Vegfb on CD diet. These data suggest that decreasing VEGF-B signaling can be used to prevent the development of the main pathologies in NASH.

FIG. 14 shows that deletion of Vegfb in mice on short-term CD diet prevented the development of hepatic inflammation as shown by reduced CD45 and F4/80 expression.

### Example 4: A Neutralizing Anti-VEGF-B Antibody Treats or Prevents Progression of Nonalcoholic Fatty Liver Disease (NAFLD) and Nonalcoholic Steatohepatitis (NASH) in Mice on Short-Term Choline-Deficient High Fat (CD) Diet

Therapeutic Anti-VEGF-B Treatment (Using 2H10) does not Impact on Body Weight, Blood Glucose Levels, Glucose Tolerance or Insulin Sensitivity

Five week old male C57BL/6 mice where fed a CD diet (Research Diets; D05010402) for 5 months. The antibody treatment commenced at week 12 and mice were treated with 2H10 or isotype-matched control antibody for 10 weeks. Animals were injected intraperitoneally (i.p.) twice weekly with a 80 μg dose of anti-VEGF-B antibody. The study also included aged and sex matched chow-fed WT mice. BW and BG levels were recorded during the trial. The food was removed for 2 h prior to BG recording. Glucose measurements were performed on blood drawn from the tail vein using a Bayer Contour Glucose meter. IPGTT and IPITT were performed after 17 weeks on CD-diet on un-starved mice or mice that had their food removed 2 h before the experiment. For the tolerance tests animals were injected intraperitoneally with 1 mg glucose per g BW (IPGTT) and with 0.75 mU insulin per g BW (IPITT).

Anti-VEGF-B treatment did not impact on body weight or glucose and insulin sensitivities in short-term CD. These data indicate that the improved glucose tolerance and insulin sensitivity observed in CD diet-fed Vegfb^{+/-} as compared to Vegfb^{-/-} mice and WT mice can be linked to developmental effects associated with the Vegfb^{-/-} mouse model.

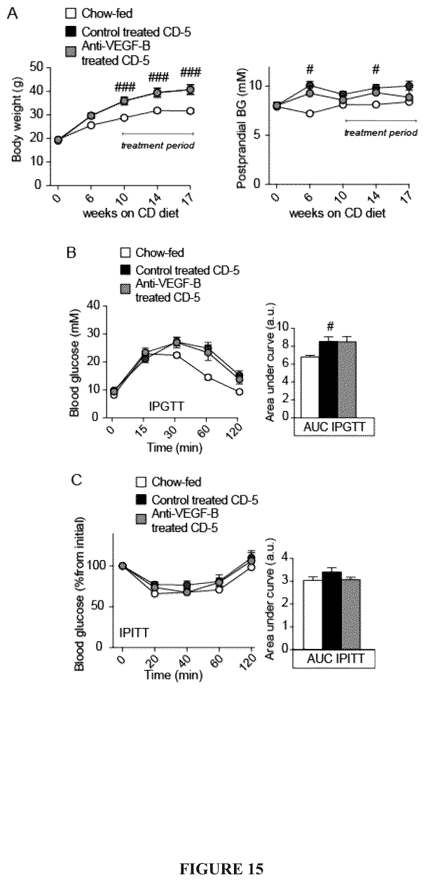

FIG. 15 shows that anti-VEGF-B treatment in mice on short-term CD diet (using 2H10) did not impact on (A) body weight, blood glucose levels, (B) glucose tolerance or (C) insulin sensitivity.

### Therapeutic Anti-VEGF-B Treatment (Using 2H10) Improves Hepatic Function

Five week old male C57BL/6 mice where fed a CD diet (Research Diets; D05010402) for 5 months. Antibody treatment commenced at week 12 and mice were treated with 2H10 or isotype-matched control antibody for 10 weeks. Animals were injected intraperitoneally (i.p.) twice weekly with a 80 μg dose of anti-VEGF-B antibody. The study also included aged and sex matched chow-fed WT. Animals were sacrificed with isofluorane anaesthetics, liver were dissected and weighed and total blood was removed by cardiac puncture. The blood was centrifuged at 14000 rpm, 4° C. for 10 minutes, serum was separated and frozen in aliquots at −80° C. ALAT serum levels were analysed at the Swedish University of Agricultural Science in Uppsala, Sweden. 2H10 therapy prevented liver damage, as measured by reduced serum ALAT levels, in anti-VEGF-B treated C57BL/6 mice on short-term CD diet.

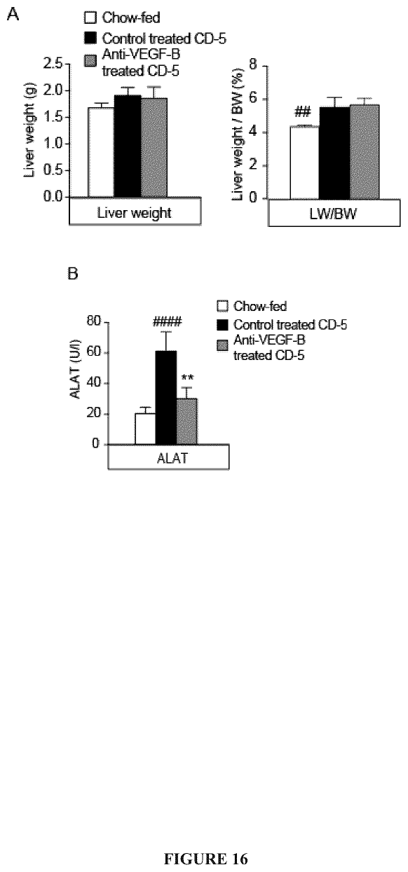

FIG. 16 shows that anti-VEGF-B treatment in mice on short-term CD diet (using 2H10) improved hepatic function as shown by liver weight and ratio of liver weight and body weight (A) and serum analysis of ALAT levels (B).

### Therapeutic Anti-VEGF-B Treatment (Using 2H10) Reduces Hepatic Lipid Accumulation

CD-fed C57BL/6 mice and aged and sex matched chow-fed WT were used for analysis. Animals received 2H10 or isotype-matched control antibody treatment as described above.

Oil red O (ORO) analysis was performed on isolated livers as previously described. By using 2H10 to reduce VEGF-B levels in C57BL/6 mice on short-term CD, liver steatosis was decreased and lipid droplets were reduced both in number and size.

Commercially available kits were used for enzymatic determination of non-esterified fatty acids (NEFAs; Wako Chemicals), beta-hydroxybutyrate (Stanbio Laboratories) and triglycerides (Sigma-Aldrich) on isolated serum. Anti-VEGF-B therapy normalized plasma levels of ketones (KBs), non-esterified fatty acids (NEFAs) and triglycerides (TGs) in mice on CD diet.

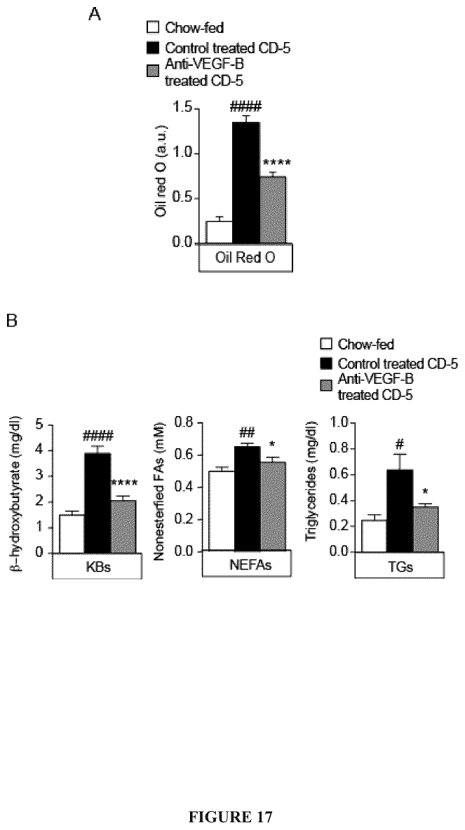

FIG. 17 shows that anti-VEGF-B treatment (using 2H10) in C57BL/6 mice on short-term CD diet reduced hepatic lipid accumulation.

### Therapeutic Anti-VEGF-B Treatment (Using 2H10) Prevents Development of Hepatic Steatosis

CD-fed C57BL/6 mice and aged and sex matched chow-fed WT were used for analysis. Animals received 2H10 or isotype-matched control antibody treatment as described above.

Immunostaining was performed on isolated livers. Briefly, animals were sacrificed with isofluorane anaesthetics, livers were dissected fixed in 4% PFA for 24 hours and subsequently processed for paraffin embedding using standard procedures and 6 μm sections were prepared. Antigen retrieval was performed using Antigen retrieval solution Ph6 (Dako #S2367) and heating at 98° C. for 10 min. Sections were incubated at 4° C. overnight with guinea pig anti-adipophilin (Fitzgerald). Before addition of appropriate fluorescently labeled secondary antibodies (Invitrogen, Alexa Fluor) samples were incubated with biotinylated donkey anti-guinea pig antibody (Jackson) for 1 hour at RT. At least 10 frames per animal stained for adipophilin or tip47 within each section were photographed with an Axio Vision microscope (Carl Zeiss) at 20× magnification. The amount of respective staining were quantified using Axio Vision Run wizard program for hepatic adipophilin staining (pixel²/μm²).

By using 2H10 to reduce VEGF-B levels in C57BL/6 mice on short-term CD the development of liver steatosis and NASH were prevented. Anti-VEGF-B antibody treatment reduced hepatic lipid content by over 50%, as compared to control treated mice on CD diet.

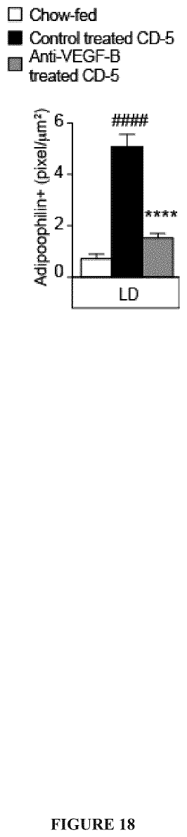

FIG. 18 shows that anti-VEGF-B treatment in C57BL/6 mice on short-term CD diet (using 2H10) prevented development of hepatic steatosis as shown by a reduction in hepatic adipophilin expression.

### Therapeutic Anti-VEGF-B Treatment (Using 2H10) Prevents the Development of Hepatic Inflammation

CD-fed C57BL/6 mice and aged and sex matched chow-fed WT were used for analysis. Animals received 2H10 or isotype-matched control antibody treatment as described above.

Immunostaining was performed on isolated livers. Briefly, animals were sacrificed with isofluorane anaesthetics, livers were dissected and flash frozen on dry ice. Liver biopsies were embedded in Tissue-Tek® (Sakura) directly on the mold of the cryostat. After embedding, 12-m sections were prepared, post-fixated in ice-cold 4% PFA for 10 minutes and thereafter immunostained for CD45 or F4/80. Briefly, sections were incubated at 4° C. for 12 h with primary rat anti-CD45 (BD bioscience) and rat anti-F4/80 (Serotec) antibodies. Appropriate fluorescently labeled secondary antibodies (Invitrogen, Alexa fluor) were applied and sections were further incubated for 1 h at RT after which they were prepared for microscopy. At least 10 frames per animal stained for CD45 or F4/80 within each section were photographed with an Axio Vision microscope (Carl Zeiss) at 20× magnification. The amount of respective staining were quantified using Axio Vision Run wizard program for hepatic i) CD45 staining (pixel²/μm²) or ii) F4/80 staining (pixel²/μm²).

Using 2H10 as anti-VEGF-B therapy in mice on CD diet decreased the amount of hepatic inflammatory cells to similar levels as in chow-fed mice. These data indicate that decreasing VEGF-B signaling (using 2H10 treatment) can be used to target the inflammatory phase in the development of NASH.

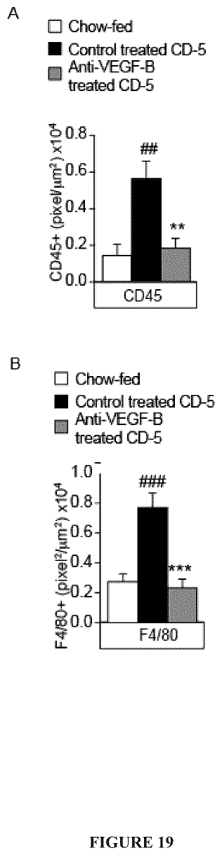

FIG. 19 shows that anti-VEGF-B treatment in mice on short-term CD diet (using 2H10) prevents the development of hepatic inflammation as shown by a reduction in (A) CD45 and (B) F4/80 expression.

### Therapeutic Anti-VEGF-B Treatment (Using 2H10) Prevents the Development of Mild Hepatic Fibrosis

CD-fed C57BL/6 mice and aged and sex matched chow-fed WT were used for analysis. Animals received 2H10 or isotype-matched control antibody treatment as described above.

Masson trichrome staining was performed on isolated livers. Briefly, animals were sacrificed with isofluorane anaesthetics. Livers were dissected, post-fixated in 4% PFA for 24 h and subsequently processed for paraffin imbedding using standard procedures. After embedding, 6-μm sections were prepared and stained with Masson trichrome (MT) (Sigma) according to the manufacturer instructions. At least 20 frames per animal stained for MT within each section were photographed with bright field microscopy (Axio Vision microscope, Carl Zeiss) at 20× magnification. The amount of fibrosis were quantified using Axio Vision Run wizard program for hepatic i) MT+ staining (pixel²/μm²).

Short-term CD can be used to induce fibrosing NASH. Fibrosing NASH is considered as the more advanced state of NASH, and is a leading cause of cirrhosis and liver-related mortality. CD diet induced mild liver fibrosis, evidenced in the vessel walls, indicative of normal extracellular matrix (ECM) deposition, together with fibrosis detected in the parenchyma, and in some cases also present in a “bridging” pattern throughout the hepatic lobule. Using 2H10 to reduce VEGF-B levels in mice on CD diet decreased the development of fibrosis, suggesting that anti-VEGF-B therapy can be used in treatment of fibrosing NASH.

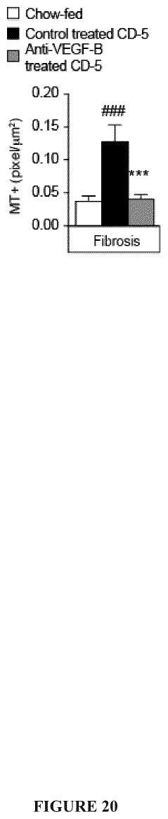

FIG. 20 shows that anti-VEGF-B treatment in mice on short-term CD diet (using 2H10) prevents the development of fibrosis using Masson trichrome staining of liver sections.

### Therapeutic Anti-VEGF-B Treatment (Using 2H10) Reduces NASH and NASH Associated Pathologies

CD-fed C57BL/6 mice and aged and sex matched chow-fed WT were used for analysis. Animals received 2H10 or isotype-matched control antibody treatment as described above.

Livers were dissected, post-fixated in 4% PFA for 24 h and subsequently processed for paraffin imbedding using standard procedures. After embedding, 6-μm sections were prepared and stained with Hematoxylin-Eosin (H&E) (Sigma) according to the manufacturer instructions. At least 20 frames per animal stained for H&E within each section were photographed with bright field microscopy (Axio Vision microscope, Carl Zeiss) at 20× magnification and analysed for the presence of degeneration of hepatocytes, formation of MDB formation, inflammatory foci and satellitosis. Total score is the amount of all identified balloon hepatocytes, MBDs, inflammatory foci and satellitosis in H&E stained liver sections.

As shown in Table 3, the livers of CD-fed mice displayed several of the characteristics of human NASH such as ballooning of hepatocytes, formation of Mallory Denck bodies, inflammatory foci and satellitosis. Therapeutic treatment with an anti-VEGF-B antibody decreased the appearance of these human NASH associated pathologies.

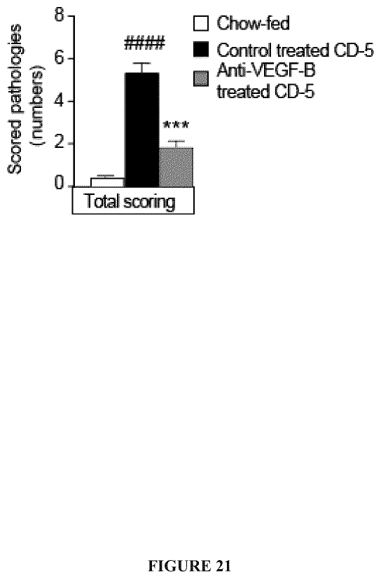

FIG. 21 shows that reducing VEGF-B levels in CD-fed mice using 2H10 antibody treatment decreased the total NASH score by more than 50%.

TABLE 3
Hepatic NASH scores in mice on short-term CD diet treated
with 2H10 or control antibody

| | Item | | | |
|---|---|---|---|---|
| | Hepatocellular | | Inflammatory | |
| Mice | ballooning | MDB | foci | Satellitosis |
| chow-fed WT | 0.2 ± 0 | 0.1 ± 0.0 | 0.1 ± 0.0 | 0.1 ± 0.0 |
| control CD diet | 3.2 ± 0.3^{####} | 0.6 ± 0.2^{#} | 1.2 ± 0.1^{####} | 0.4 ± 0.1^{#} |
| anti-VEGF-B | 1.2 ± 0.2**** | 0.3 ± 0.1* | 0.2 ± 0.1 | 0.1 ± 0.0 |
| CD diet | | | | |

Results are expressed as mean s.e.m. Statistical analyses were performed using one-way ANOVA.
^{#}P < 0.05,
^{###}P < 0.001,
^{####}P < 0.0001 compared to chow-fed WT.
*P < 0.05,
***P < 0.001,
****P < 0.0001 compared to control treated mice on CD diet.
Animal numbers were for chow-fed WT (n = 5), control treated mice on CD diet (n = 8) and anti-VEGF-B treated mice on CD diet (n = 7).

### Example 5: A Neutralizing Anti-VEGF-B Antibody Treats or Prevents Progression of Nonalcoholic Fatty Liver Disease (NAFLD) and Nonalcoholic Steatohepatitis (NASH) in Mice on Long-Term CD Diet

Therapeutic Anti-VEGF-B Treatment (Using 2H10) does not Impact on Body Weight, Postprandial Blood Glucose Levels, Glucose Tolerance or Insulin Sensitivity

Five week old male C57BL/6 mice were fed a CD diet (Research Diets; D05010402) for 12 months (CD12). The antibody treatment commenced at 32 weeks of age and mice were treated with 2H10 or isotype-matched control antibody for 20 weeks. Animals were injected intraperitoneally (i.p.) twice weekly with a 80 μg dose of anti-VEGF-B antibody. The study also included aged and sex matched chow-fed WT. BW and BG levels were recorded during the trial. The food was removed for 2 h prior to BG recording. Glucose measurements were performed on blood drawn from the tail vein using a Bayer Contour Glucose meter. IPGTT and IPITT were performed after 17 weeks on CD-diet on un-starved mice or mice that had their food removed 2 h before the experiment. For the tolerance tests animals were injected intraperitoneally with 1 mg glucose per g BW (IPGTT) and with 0.75 mU insulin per g BW (IPITT).

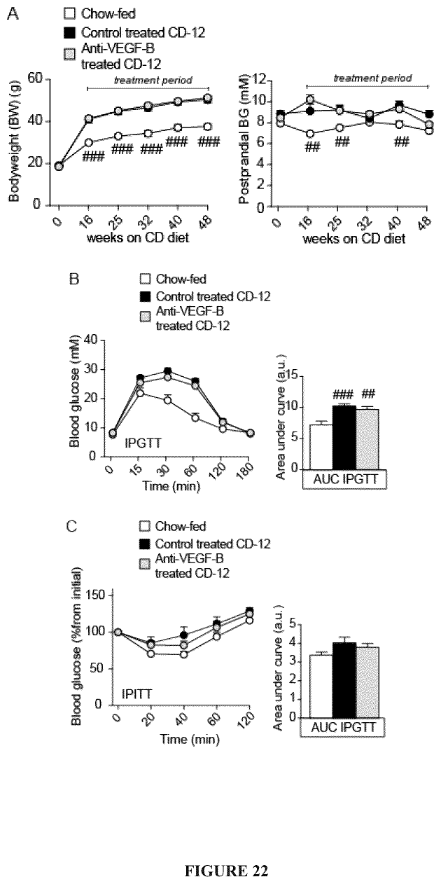

As shown in FIG. 22, anti-VEGF-B treatment did not impact bodyweight (A) or glucose and insulin sensitivities (B and C) by long-term CD diet in C57BL/6 mice.

### Therapeutic Anti-VEGF-B Treatment (Using 2H10) Improves Liver Function and Ameliorates the Development of Hepatocellular Carcinoma

12-month CD-fed C57BL/6 mice and aged and sex matched chow-fed WT were used for analysis. Animals received 2H10 or isotype-matched control antibody treatment as described above.

Magnetic Resonance Imaging (MRI) data was acquired using horizontal bore, 9.4 T scanner (Agilent, Yarnton, UK) equipped with a millipede transmit/receive volume coil with an inner diameter of 40 mm (Agilent, Yarnton, UK). For the MRI analysis, animals were injected with contrast agent, Primovist. The 250 mM Primovist solution (Bayer Pharma, Berlin, Germany) was diluted to 12.5 mM with saline (9 mg/ml). A bolus of 2 ul per gram body weight was delivered to the mice, while located at the isocenter, through a tail vein catheter connected via a 2 m polyetylen (PE-20) hose connected to an infusion pump positioned near the bore. For MRI scanning, T1-weighted images with fat-saturation (Fast spin echo data etl 4, kzero=1 matrix 256×192, effective time to echo=7.01 ms, 30 contiguous slices of 1 mm thickness, field of view 35.2×26.4 mm2. 15 slices were consecutively excited in each expiration period, resulting in a recovery time of twice the respiration period. The respiration period was 750-950 ms and was controlled by the isoflurane level (1.6-2.5%). Each 3d dataset took approximately 1.5 min to acquire. One dataset was acquired before the bolus, followed by 5 consecutive datasets post bolus. The intensity of healthy liver was increased within 3 minutes by the contrast agent, while the intensity from tumors was not enhanced by the contrast agent and tumors appeared hypointense post contrast.

After MRI analysis, the animals were sacrificed with isofluorane anaesthetics, the liver dissected and weighed and total blood was removed by cardiac puncture.

Long-term CD led to liver hypertrophy, reduced liver function and hepatocellular carcinoma development in C57BL/6 mice. MRI in combination with gadolidium-enhanced contrast scans can be used to visualize liver tumors. The most abundant sites for tumor development in the liver were caudate process and right lateral lobe. After 12 months on CD diet, 50% of the control Ig-treated C57BL/6 mice developed hepatocellular carcinoma, and no tumors were detected in 2H10 treated mice. Furthermore, anti-VEGF-B therapy prevented hypertrophy and improved liver function, suggesting that anti-VEGF-B therapy can be used to prevent the development of hepatocellular carcinoma.

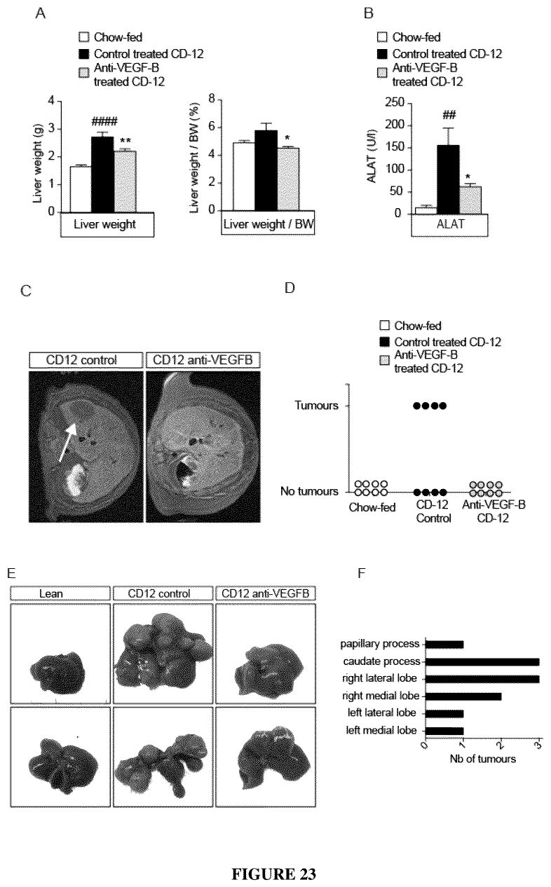

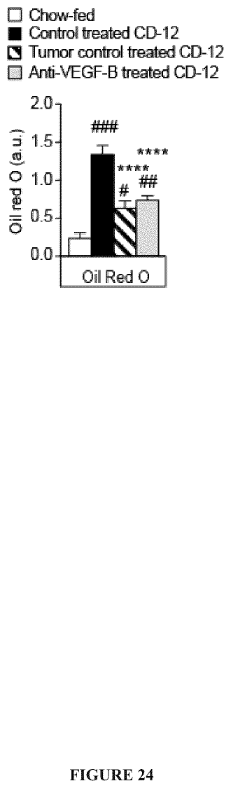

FIG. 23 shows that anti-VEGF-B treatment improved liver function and ameliorated the development of hepatocellular carcinoma.

### Therapeutic Anti-VEGF-B Treatment (Using 2H10) Reduces Hepatic Lipid Accumulation

12-month CD-fed C57BL/6 mice and aged and sex matched chow-fed WT were used for analysis. Animals received 2H10 or isotype-matched control antibody treatment as described above.

Oil red O (ORO) analysis was performed on isolated livers. Briefly, livers were dissected and flash frozen on dry ice and liver biopsies embedded in Tissue-Tek® (Sakura) directly on the mold of the cryostat. Cryosections (12 μm) were immersed 5 min in oil red O working solution (2.5 g oil red O (Sigma-Aldrich), dissolved in 400 ml 99% isopropanol, further diluted 6:10 in H2O, filtered through a 22 μm filter (Corning). Thereafter the sections were submerged for 3 s in hematoxylin solution followed by submerging in LiCO3 and rinsed for 10 min under running tap water before they were mounted. Stained sections were examined with bright field microscopy. At least 10 frames per animal stained for ORO and hematoxylin within each section were photographed with an Axio Vision microscope (Carl Zeiss) at 20× magnification. The amounts of lipid droplets were quantified using Axio Vision Run wizard program for liver ORO staining (pixel²/μm²).

By using 2H10 to reduce VEGF-B levels in mice on long-term CD diet liver steatosis can be decreased and lipid droplets were reduced both in number and size. Liver lipid content in mice on CD diet mice was reduced by more than 50% by 2H10 therapy.

FIG. 24 shows that hepatic lipid accumulation as measured by ORO staining was reduced following anti-VEGF-B treatment (using 2H10) in mice on long-term CD diet.

### Therapeutic Anti-VEGF-B Treatment (Using 2H10) Prevents the Development of Hepatic Inflammation

12-month CD-fed C57BL/6 mice and aged and sex matched chow-fed WT were used for analysis. Animals received 2H10 or isotype-matched control antibody treatment as described above.

Immunostaining for CD45 and F4/80 was performed on isolated livers. Briefly, livers were dissected and flash frozen on dry ice. Liver biopsies were embedded in Tissue-Tek® (Sakura) directly on the mold of the cryostat. After embedding, 12-μm sections were prepared, post-fixated in ice-cold 4% PFA for 10 minutes and thereafter immunostained for CD45 or F4/80. Briefly, sections were incubated at 4° C. for 12 h with primary rat anti-CD45 (BD bioscience) and rat anti-F4/80 (Serotec) antibodies. Appropriate fluorescently labeled secondary antibodies (Invitrogen, Alexa fluor) were applied and sections were further incubated for 1 h at RT after which they were prepared for microscopy. At least 10 frames per animal stained for CD45 or F4/80 within each section were photographed with an Axio Vision microscope (Carl Zeiss) at 20× magnification. The amount of respective staining were quantified using Axio Vision Run wizard program for hepatic i) CD45 staining (pixel²/μm²) or ii) F4/80 staining (pixel²/μm²).

Long term CD led to increased liver inflammation in C57BL/6 mice. The inflammatory response was increased in control treated mice on CD diet, both with and without tumors, with approximately 2 and 5 fold, respectively, as compared to chow-fed mice. Using 2H10, to reduce VEGF-B levels, in mice on CD diet normalized the amount of hepatic inflammatory cells. Thus, the data suggests that decreasing VEGF-B signaling (using 2H10 treatment) efficiently targets the inflammatory phase in the development of NASH/hepatocellular carcinoma.

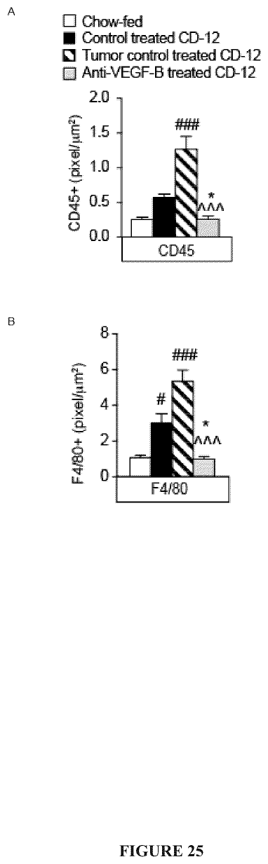

FIG. 25 shows that anti-VEGF-B treatment in mice on long-term CD diet prevents the development of hepatic inflammation as shown by a reduction in CD45 and F4/80 expression.

1. A method for treating or preventing progression of severe hepatic steatosis or nonalcoholic steatohepatitis (NASH) or for preventing or reducing the risk of developing a complication thereof in a subject suffering from severe hepatic steatosis or nonalcoholic steatohepatitis (NASH), the method comprising administering to the subject a compound that inhibits vascular endothelial growth factor B (VEGF-B) signaling, wherein the compound that inhibits VEGF-B signaling is a protein comprising an antibody variable region that binds to or specifically binds to VEGF-B and neutralizes VEGF-B signaling.

2. (canceled)

3. The method of claim 1, wherein the complication thereof is hepatic fibrosis or cirrhosis.

4. (canceled)

5. The method of claim 1, wherein the subject suffering from the severe hepatic steatosis or NASH is additionally overweight or obese and/or suffers from diabetes and/or suffers from metabolic syndrome.

6.-7. (canceled)

8. The method of claim 1, wherein the compound is administered in an amount effective to have one or more of the following effects:
a) reduce or prevent lipids, including neutral lipids accumulating in the liver of a subject;
b) reduce or prevent inflammation in the liver of the subject;
c) reduce or prevent development of pathologic changes of severe hepatic steatosis or NASH in the liver of a subject;
d) reduce or prevent hepatic fibrosis and/or cirrhosis
e).

9. A method for reducing the level of lipids or for reducing inflammation in the liver of a subject suffering from severe hepatic steatosis or nonalcoholic steatohepatitis (NASH), the method comprising administering to the subject a compound that inhibits VEGF-B signaling, wherein the compound that inhibits vascular endothelial growth factor B (VEGF-B) signaling is a protein comprising an antibody variable region that binds to or specifically binds to VEGF-B and neutralizes VEGF-B signaling.

10.-13. (canceled)

14. The method of claim 1, wherein the compound is a protein comprising a Fv.

15. The method of claim 14, wherein, the protein is selected from the group consisting of:
(i) a single chain Fv fragment (scFv);
(ii) a dimeric scFv (di-scFv);
(iii) a diabody;
(iv) a triabody;
(v) a tetrabody;
(vi) a Fab;
(vii) a F(ab′)₂;
(viii) a Fv;
(ix) one of (i) to (viii) linked to a constant region of an antibody, Fc or a heavy chain constant domain (C_{H}) 2 and/or C_{H}3; and
(x) an antibody.

16. (canceled)

17. The method of claim 1, wherein the compound competitively inhibits the binding of antibody 2H10 to VEGF-B, wherein antibody 2H10 comprises a heavy chain variable region (V_{H}) comprising a sequence set forth in SEQ ID NO: 3 and a light chain variable region (V_{L}) comprising a sequence set forth in SEQ ID NO: 4.

18. The method of claim 1, wherein the compound is a protein comprising a humanized variable region of antibody 2H10.

19. (canceled)

20. The method of claim 1, wherein the compound is a protein comprising:
(i) a V_{H} comprising:
(a) a CDR1 comprising a sequence set forth in amino acids 25-34 of SEQ ID NO: 3;
(b) a CDR2 comprising a sequence set forth in amino acids 49-65 of SEQ ID NO: 3; and
(c) a CDR3 comprising a sequence set forth in amino acids 98-108 of SEQ ID NO: 3; and
(ii) a V_{L} comprising:
(a) a CDR1 comprising a sequence set forth in amino acids 23-33 of SEQ ID NO: 4;
(b) a CDR2 comprising a sequence set forth in amino acids 49-55 of SEQ ID NO: 4; and
(c) a CDR3 comprising a sequence set forth in amino acids 88-96 of SEQ ID NO: 4.

21. The method of claim 20, wherein the protein is an antibody comprising a V_{H} comprising a sequence set forth in SEQ ID NO: 5 and a V_{L} comprising a sequence set forth in SEQ ID NO: 6.

22. A method for treating or preventing progression of severe hepatic steatosis or nonalcoholic steatohepatitis (NASH) or for preventing or reducing the risk of developing a complication thereof in a subject suffering from severe hepatic steatosis or nonalcoholic steatohepatitis (NASH), the method comprising administering to the subject a compound that inhibits vascular endothelial growth factor B (VEGF-B) signaling, wherein the compound that inhibits VEGF-B signaling is a nucleic acid that inhibits or prevents expression of VEGF-B.

23. The method of claim 22, wherein the nucleic acid is selected from the group consisting of an antisense nucleic acid, a siRNA, a ribozyme and a DNAzyme.
